(19) United States
(12) Patent Application Publication
(10) Pub. No.: US 2024/0417514 A1
(43) Pub. Date: Dec. 19, 2024

(54) HIGH-PERFORMANCE BENZOXAZINE DERIVATIVES VITRIMERS

(71) Applicant: Luxembourg Institute of Science and Technology (LIST), Esch-sur-Alzette (LU)

(72) Inventors: Pierre Verge, Esch-sur-Alzette (LU); Laura Puchot, Esch-sur-Alzette (LU); Daniel Schmidt, Esch-sur-Alzette (LU); Antoine Adjaoud, Esch-sur-Alzette (LU); Henri Perrin, Esch-sur-Alzette (LU)

(21) Appl. No.: 18/698,954

(22) PCT Filed: Oct. 6, 2022
(86) PCT No.: PCT/EP2022/077812

§ 371 Date: Apr. 5, 2024

(30) Foreign Application Priority Data
Oct. 7, 2021 (LU) 500714

(51) Int. Cl. C08G 73/02; C07D 413/12; C07D 413/14

(52) U.S. Cl. CPC C08G 73/0233; C07D 413/12; C07D 413/14

(57) ABSTRACT

The invention also relates to a process for synthesizing an ester-containing benzoxazine monomer of formula (1) comprising the following steps consisting of:
c) reacting a phenolic acid derivative of formula (II), comprising at least one R*** group on the phenolic ring:

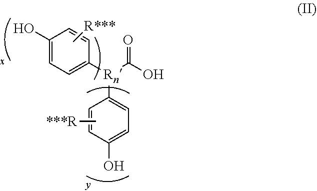

wherein x is of from 0 to 1, and y=1-x,
with a polyfunctional molecule or oligomer of formula (III)

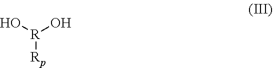

at a temperature of from 25° C. to 200° C., during 1 h-72 h, in the presence of a catalyst of Bronsted acid type, resulting in a phenol terminated oligomer or molecule (compound (IV)), and reacting the compound (IV) with a mixture of:
   an amino-alcohol of formula (V):

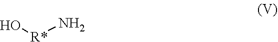

a primary amine derivative of formula (VI),

R**—NH₂ (VI), and
paraformaldehyde of formula (VII)

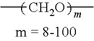

at a temperature range of from 80° C. to 100° C., from 1 h to 10 h, under stirring, for obtaining the compound of formula (I);

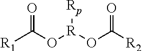

The invention is directed to the field of ester-containing benzoxazine derivatives vitrimers and to a process of manufacturing thereof and the use of said vitrimers in various applications.

### TECHNICAL FIELD

Composites are almost all the cases produced from thermoset resins, a material of choice for numerous applications because of their dimensional stability, mechanical properties and creep/chemical resistance. However, as a result of their permanent molecular architecture, they are impossible to recycle or to reprocess, and ends up in landfills.

A chemical way to tackle this drawback is offered by the introduction of exchangeable chemical bonds, leading to dynamic cross-links. Polymer networks containing such exchangeable bonds are also known as covalent adaptable networks (CANs) (W. Denissen et al.—Wim Denissen, Johan M. Winne and Filip E. Du Prez, Chem. Sci., 2016, 7, 30-38). CANs may be further classified into two groups depending on their exchange mechanism, either dissociative or associative. In the first, chemical bonds are first broken and then formed again at another place. Diels Alder reactions are the most common mechanism of dissociative CANs. In the second, polymer networks do not depolymerise upon heating, but are characterized by a fixed cross-link density. Covalent bonds are only broken when new ones are formed, making these networks permanent as well as dynamic. The first reported associative CANs (2005) were based on photo-mediated reactions by using allyl sulfides for instance. Later, a similar exchange mechanism was introduced by using alternative radical generators with trithiocarbonates. In 2011, Leibler et al. (D. Montamal, M. Capelot, F. Tournilhac and L. Leibler, Science, 2011, 334, 965-968) extended the field of associative CANs by adding a suitable transesterification catalyst to epoxy/acid or epoxy/anhydride polyester-based networks, resulting in permanent polyester/polyol networks that show a gradual viscosity decrease upon heating. Such a distinctive feature of vitreous silica had never been observed in organic polymer materials. Hence, the authors introduced the name vitrimers for those materials.

Vitrimers are portrayed as the third class of polymeric material owing to their outstanding features. The dynamic nature of the covalent network, arises from reversible chemical bonds, allows the material to be healed, recycled and reprocessed like thermoplastics. These exchange reactions are triggered by external stimulus, most frequently temperature. The viscosity of vitrimers gradually decreased upon heating providing malleability to the network while permitting internal stress to relax. Network integrity over the entire range of application ensures mechanical and solvent resistance.

Following the prototypal vitrimer developed by Leibler et al. in 2011 (previously mentioned), dynamic transesterification reactions demonstrated extensive interest over the last decade. These chemical exchanges induced at elevated temperatures between ester linkages and hydroxyl groups are responsible for topology rearrangements. Transesterification mechanism was implemented in cross-linked network to design self-healable, recyclable and reprocessable material with tunable properties.

Demongeot et al. (A. Demongeot, R. Groote, H. Goossens, T. Hoeks, F. Tournilhac and L. Leibler, Macromolecules, 2017, 50 (16), 6117-6127) adapted the vitrimer concept to commercially available thermoplastic. Cross-linked polybutylene terephthalate (PBT) vitrimer based on transesterification exchanges was successfully prepared by reactive extrusion. In addition to improving the manufacturing techniques and the potential scope of these networks, global environmental context urges the scientific community to promote sustainable polymer derived from naturally occurring feedstocks. Altuna et al. (F. I. Altuna, V. Pettarin and R. Williams, Green Chem., 2013, 15, 3360-3366) endeavoured to generate fully bio-based polyester showing properties reminiscent of vitrimers, starting from epoxidized soybean oil and an aqueous citric acid solution.

Furthermore, Legrand et al. (A. Legrand and C. Soulié-Ziakovic, Macromolecules, 2016, 49, 5893-5902) enabled to extend the scalability of applications of vitrimer networks by developing a silica-reinforced epoxy vitrimer nanocomposites with enhanced properties.

Polybenzoxazines are a new type of thermoset with outstanding mechanical and thermal properties. As many other thermosets, they cannot be reshaped, re-processed nor recycled. A few examples have been reported showing a reasonable level of healability (L. Zhang, Z. Zhao, Z. Dai, L. Xu, F. Fu, T. Endo, X. Liu, ACS Macro. Lett. 2019, 8, 5, 506-511 and Arslan M., Kiskan B., Y. Yagci, Sci. Rep. 2017, 7, 5207). However, polybenzoxazine remains a class of high performance materials without any demonstration of vitrimers capabilities. Such sustainable vitrimer will widespread the use of polybenzoxazine towards smart coatings, reversible adhesives, or even recyclable matrix resins for composite materials.

### DISCLOSURE OF THE INVENTION

The invention has for technical problem to provide a solution to at least one drawback of the above cited prior art.

The invention relates to an ester containing benzoxazine monomer of formula (I)

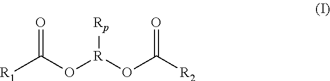

wherein
R is selected from the group consisting of a linear or branched C₁-C₂₀ alkyl group, preferably a C₁-C₁₂ alkyl group, more preferably a C₁-C₆ alkyl group, optionally including an heteroatom, a cyclo(C₃-C₆alkyl) group, a heterocyclo(C₃-C₆alkyl) group, wherein the heteroatom is selected from the group consisting of from N, S, Si and O, a linear or branched C₂-C₁₂, preferably a C₂-C₆, alkenyl group, substituted or unsubstituted linear or branched C₂-C₁₂, preferably a C₂-C₆, alkynyl group, a linear or branched C₁-C₆ alkyl or C₂-C₆ alkenyl substituted or unsubstituted phenyl group and a —(CH₂)ₙ₃-phenyl group, wherein n3 is an integer from 1 to 10, a siloxane group;
R₁ is

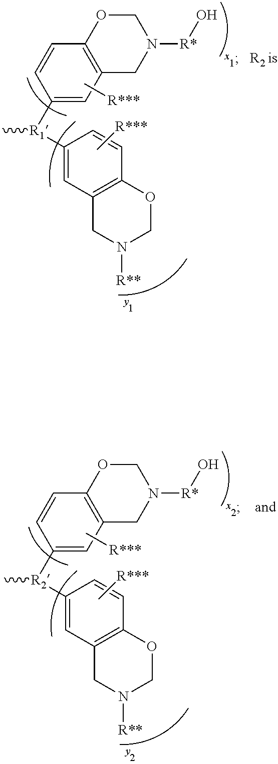

Rₚ is selected from the group consisting of H, a linear or branched C₁-C₂₀, preferably C₁-C₁₂, alkyl or alkoxy group, a linear or branched C₂-C₁₂, preferably C₂-C₆, alkenyl or alkylenoxy group, a substituted or unsubstituted linear or branched C₂-C₁₂, preferably C₂-C₆, alkynyl group, a linear or branched C₁-C₆, preferably C₁-C₄, alkyl or C₂-C₆, preferably C₂-C₄, alkenyl substituted or unsubstituted phenyl group and

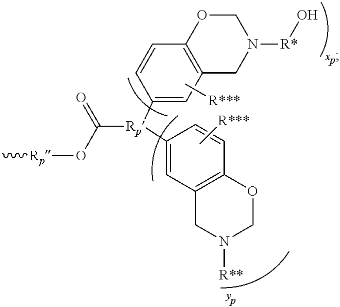

wherein
R₁ and R₂ of formula (I) are different;
x1, x2 and xₚ, independently, are of from 0 to 1; y₁=1−x₁; y₂=1−x₂ yₚ=1−xₚ, with the proviso that x₁, x₂ and xₚ are not together 0;
p is 1-100;
R₁′, R₂′, and Rₚ′, independently, are selected from the group consisting of a C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
R* is selected from the group consisting of a linear or branched C₁-C₆ alkyl or alkoxy group, a cyclo(C₃-C₆ alkyl) group, a heterocyclo(C₃-C₆alkyl) group, wherein the hetero atom is selected from the group consisting of N, Si, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group and a —(CH₂)ₙ₃-phenyl group, —(CH₂)ₙ₃—O—(CH₂)ₙ₄, wherein n3 and n4, independently, are an integer from 1 to 10; R** is the same as R* and further includes a member selected from a O—, N—, Si— or S—(CH₂)ₙ₃—CH—(CH₃)₂ group, a O—, N—, Si— or S—(CH₂)ₙ₃—(CHZ)ₙ₄—(CH₃)₂ group, a O—, N—, Si— or S—(CH₂)ₙ₃—(CHZ)ₙ₄—(CH₂)ₙ₃—CH₃ group, a O—, N—, Si— or S—(CHZ)ₙ₄—(CH₂)ₙ₃—CH₃ group, a O—, N—, Si— or S—(CHZ)ₙ₄—[(CH₂)ₙ₃—CH₃]₂ group and O-substituted or unsubstituted C₂-C₆ linear or branched alkynyl group, —(CH₂)ₙ₃—C≡N, a polycyclic aromatic or heteroaromatic hydrocarbon, such as naphthalene, anthracene, fluorene, phenanthrene, optionally substituted by a linear or branched C₁-C₆ alkyl or alkoxy group, cyclo(C₃-C₆ alkyl), heterocyclo(C₃-C₆ alkyl), wherein the hetero atom is selected from the group consisting of N, S, Si, and O, linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or an alkylenoxy group and a linear or branched C₁-C₆ alkyl or C₂-C₆ alkenyl substituted or unsubstituted phenyl group, and at least one O atom is present or not between two adjacent C,
R*** is selected from the group consisting of H, OH and a O-linear or branched C₁-C₆ alkyl group, and further includes a linear or branched C₁-C₁₅ alkyl group or a C₂-C₁₅ alkenyl group or

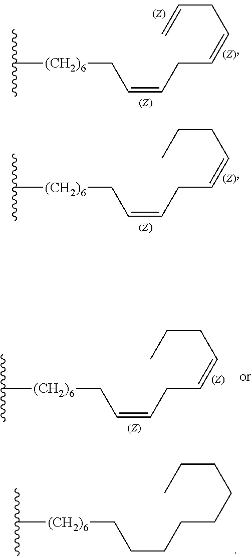

The ester-containing benzoxazine monomer of the invention is advantageously suited for obtaining polybenzoxazine derivatives vitrimers by a polymerization involving the benzoxazine ring opening and a self-polymerisation under heat, resulting to said polybenzoxazine derivatives vitrimers. Owing to the specific monomer starting product, the vitrimers of the invention exhibit self-healing, reshaping, reprocessability and recycling properties. For the rest of the document, benzoxazine vitrimers will always refer to the polymerized form of the ester-bond benzoxazine monomers.

The polybenzoxazine derivatives vitrimers properties are tightly connected to the properties of the ester-containing benzoxazine monomer.

As may be seen from formula (I), the monomer includes a benzoxazine ring moiety that allows the cross-linking of said monomer upon heating and that promotes the reprocessing of the obtained benzoxazine vitrimers thanks to the exchangeable ester bonds it forms once crosslinked. Benzoxazine gives thermosetting properties such as high-temperature and flammability performance, high strength, thermal stability, low water absorption, chemical resistance, low melt viscosities, and near-zero shrinkage.

The presence of a moiety consisting of ester bonds and free aliphatic hydroxyl groups are essential to form a dynamic and reversible network of the benzoxazine derivatives vitrimers, allowing the material to be recycled, reshaped and reprocessed. An amine terminated with a hydroxyl group allows to close the oxazine ring and allows the transesterification reactions. Accordingly, the essential features of the monomer of the invention rely on the benzoxazine-containing moiety, ester bonds and free aliphatic hydroxyl groups. The Tg of such polybenzoxazine may be of from 25° C. to 300° C.

x₁, x₂ and xₚ, values, independently, may be of from 0 to 1, with the proviso that x₁, x₂ and xₚ are not together 0, and y₁, y₂, and yₚ values are, respectively and independently, 1-x₁, 1-x₂ and 1-xₚ, more preferentially from 0.5 to 1.

Preferably, R* is selected from the group consisting of a linear or branched C₁-C₄ alkyl or alkoxy group, a linear or branched C₂-C₄ alkenyl or alkylenoxy group, an unsubstituted linear or branched C₂-C₄ alkynyl group, an unsubstituted phenyl group and a —(CH₂)ₙ₃-phenyl group, —(CH₂)ₙ₃—O—(CH₂)ₙ₄, wherein n3 and n4, independently, are an integer from 1 to 6;
More preferably, R* may be selected from the group consisting of —CH₃, —(CH₂)ₙ₃—CH₃, —(CH₂)ₙ₃—CH—[(CH₂)ₙ₄—CH₃]₂, —C(CH₃)₃, —(CH₂)ₙ₃—(C₆H₅), —(CH₂)ₙ₃—CH═CH₂, —(CH₂)ₙ₃—C≡CH, —(CH₂)ₙ₃—O—(CH₂)ₙ₄ wherein n3 and n4 independently are integer from 1 to 4, phenyl, and —(CH₂)₃-phenyl.

Preferably, R** is the same as R* and may further include a member selected from O—, N—, Si— or S—(CH₂)ₙ₃—CH—(CH₃)₂ group, a O—, N—, Si— or S—(CH₂)ₙ₃—(CHZ)ₙ₄—(CH₃)₂ group, a O—, N—, Si— or S—(CH₂)ₙ₃—(CHZ)ₙ₄—(CH₂)ₙ₃—CH₃ group, a O—, N—, Si— or S—(CHZ)ₙ₄—(CH₂)ₙ₃—CH₃ group, a O—, N—, Si— or S—(CHZ)ₙ₄—[(CH₂)ₙ₃—CH₃]₂ group and O-substituted or unsubstituted C₂-C₄ linear or branched alkynyl group, —(CH₂)ₙ₃—C≡N, cyclo(C₃-C₄ alkyl), heterocyclo(C₃-C₄ alkyl), polycyclic aromatic or heteroaromatic hydrocarbon, wherein the hetero atom is selected from the group consisting of N, S, Si, and O, such as naphthalene, anthracene, fluorene, furane, which may optionally be substituted by a linear or branched C₁-C₄ alkyl or alkoxy group, a linear or branched C₂-C₄ alkenyl or alkylenoxy group, or by a substituted or unsubstituted linear or branched C₂-C₄ alkynyl group, wherein n3 and n4, independently, are an integer from 1 to 6, Z being as above defined.

More preferably, R** can be the group R*, or may be selected from the group consisting of —CH₃, —(CH₂)ₙ₃—CH₃, —(CH₂)ₙ₃—CH—[(CH₂)ₙ₄—CH₃]₂, —C(CH₃)₃, —(CH₂)ₙ₃—(C₆H₅), —(CH₂)ₙ₃—CH═CH₂, —(CH₂)ₙ₃—C≡CH, O—(CH₂)ₙ₃—C≡CH, O—(CH₂)ₙ₃—C≡N, (CH₂)ₙ₃—C≡N, and —(CH₂)ₙ₃-substituted or unsubstituted furan, phenyl, and wherein n3 and n4, independently, are integer from 1 to 4.

R*** may be selected from the group consisting of H, OH and a O-linear or branched C₁-C₄ alkyl group, and may further include linear or branched C₁-C₁₀ alkyl group or C₂-C₁₀ alkenyl group or

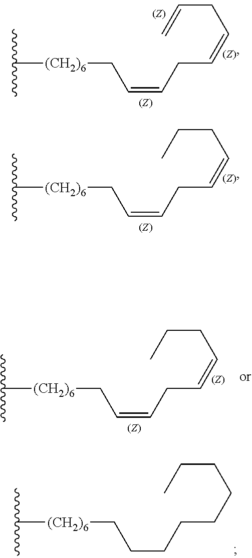

R*** may preferably be selected from the group consisting of H, OH and a O-linear or branched C₁-C₃ alkyl group, and may further include a linear or branched C₁-C₆ alkyl group or a C₂-C₆ alkenyl group or

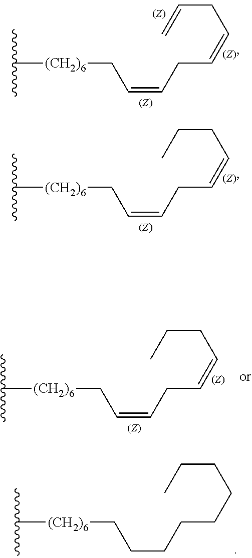

More preferably R*** is H.

The expression “substituted” as defined above, relates to the presence of some linear or branched alkyl groups in C₁-C₆.

The invention also relates to a process for synthesizing an ester-containing benzoxazine monomer of formula (I) comprising the following steps consisting of:
a) reacting a phenolic acid derivative of formula (II), comprising at least one R*** group on the phenolic ring:

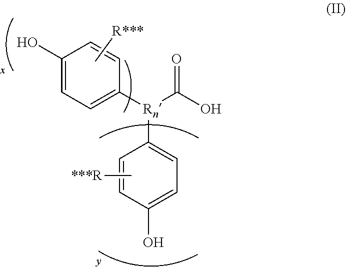

wherein x is of from 0 to 1, and y=1−x,
with a polyfunctional molecule or oligomer of formula (III)

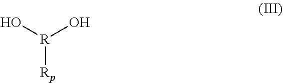

at a temperature of from 25° C. to 200° C., during 1 h-72 h, in the presence of a catalyst of Bronsted acid type, resulting in a phenol terminated oligomer or molecule (compound (IV)), and
b) reacting the compound (IV) with a mixture of:
   an amino-alcohol of formula (V):

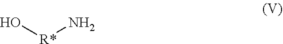

a primary amine derivative of formula (VI),

R**—NH₂ (VI), and
paraformaldehyde of formula (VII)

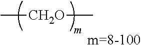

at a temperature range of from 80° C. to 100° C., from 1 h to 10 h, under stirring, for obtaining the compound of formula (I);
wherein R₁, R₂, R₁¹, R₂′, Rₚ, R*, R**, R***, x₁, x₂, xₚ, y₁, y₂, yₚ, and p are, independently, as defined above, Rₙ′ being R₁′ or R₂, R₁′ being different of R₂′, with the proviso that when at least one R*** of the phenolic acid derivative is in ortho position with regard to —OH group, then R*** is H.
x₁, x₂, xₚ and y₁, y₂, yₚ represent the proportion between benzoxazine groups when prepared from an aminoalcohol and the other amine(s). In other words, x₁, x₂, xₚ and y₁, y₂ and yₚ can be defined as

$${x}_{1} = \frac{{n}_{aminoalcohol ( R 1 )}}{{n}_{amines ( R 1 )}^{total}}$$

$${x}_{2} = \frac{{n}_{aminoalcohol ( R 2 )}}{{n}_{amines ( R 2 )}^{total}}$$

$${x}_{p} = \frac{{n}_{aminoalcohol ( Rp )}}{{n}_{amines ( Rp )}^{total}}$$

$${y}_{1} = \frac{{n}_{aminoalcohol ( R 1 )}}{{n}_{amines ( R 1 )}^{total}}$$

$${y}_{2} = \frac{{n}_{aminoalcohol ( R 2 )}}{{n}_{amines ( R 2 )}^{total}}$$

$${y}_{p} = \frac{{n}_{aminoalcohol ( Rp )}}{{n}_{amines ( Rp )}^{total}}$$

wherein n_{amine(R1)}^{total}=n_{amines(R1)}+n_{aminoalcohol(R1)}, and n_{aminoalochol(R1)} being the number of aminoalcohol per R₁ group, n_{amines(R1)} represents the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R2)} being the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} being the number of aminoalcohol per Rₚ group, n_{amines(Rp)} represents the number of amines (excepting the number of aminoalcohol) per group Rₚ and n_{amine(Rp)}^{total}+n_{amines(Rp)}+n_{aminoalcohol(Rp)} is the total number of amino groups per group Rₚ.

The ester-containing benzoxazine monomer of the invention is advantageously suited for obtaining polybenzoxazine derivatives vitrimers by a polymerization involving the benzoxazine ring opening and a self-polymerisation under heat.

The Applicant has shown that the specific starting reactants are providing an ester-containing benzoxazine monomer, which in turn, after polymerization, is giving the polybenzoxazine derivatives vitrimers comprising polymerized benzoxazine.

The benzoxazine ring, obtained from the reaction of the specific compounds ((II)—(VII)) which allows the material to be cross-linked (processed) upon heating, helps the reprocessing thanks to the exchangeable and reversible ester bonds, and free aliphatic hydroxyl groups. Also, the benzoxazine ring moiety gives thermosetting properties such as high-temperature and flammability performance, high strength, thermal stability, low water absorption, chemical resistance, low melt viscosities, and near-zero shrinkage.

The phenolic acid derivative (formula (II)) may be more preferably selected from the group consisting of mono-, di-, tri-hydroxybenzoic acid derivatives, anacardic acid derivatives, hydroxycinnamic acid derivatives, aliphatic X-hydroxyphenyl acid derivatives, wherein X is 2-4, and aliphatic diphenolic acid derivatives, or mixtures thereof.

Most preferred aliphatic mono-, di-, tri-hydroxybenzoic acid derivatives may be of formula (VIII)

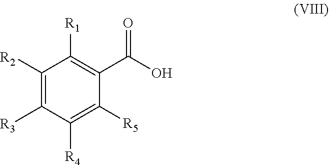

wherein R′ is omitted, and the R₁ to R₅ groups corresponding to R***, and one among R₁-R₅ is a hydroxyl group, then at least one H is in phenolic ortho-position, the rest being defined above.

Especially, in formula (VIII), at least one combination of R₁ to R₅ may be selected from the group consisting of:
R₁=OH, R₂=H, R₃=R₄=R₅=H or CH₃ or CH₂—CH₃ or CH₂—CH₂CH₃ or CH₂—CH(CH₃)₂,
R₂=OH, R₁=R₃=H, R₄=R₅=H or CH₃ or CH₂—CH₃ or CH₂—CH₂CH₃ or CH₂—CH(CH₃)₂,
R₃=OH, R₂=R₄=H, R₁=R₅=H or CH₃ or CH₂—CH₃ or CH₂—CH₂CH₃ or CH₂—CH(CH₃)₂,
R₄=OH, R₃=R₅=H, R₁=R₂=H or CH₃ or CH₂—CH₃ or CH₂—CH₂CH₃ or CH₂—CH(CH₃)₂,
R₅=OH, R₁=H, R₂=R₃=R₄=H or CH₃ or CH₂—CH₃ or CH₂—CH₂CH₃ or CH₂—CH(CH₃)₂.

Most preferred anacardic acid derivatives may be of formula (IX),

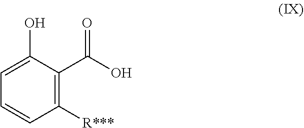

wherein R′ is omitted, and R*** is

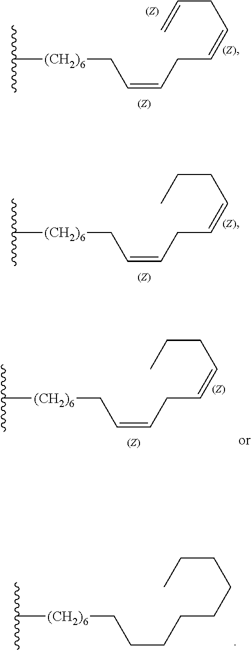

Most preferred hydroxycinnamic acid derivatives may be of formula (X)

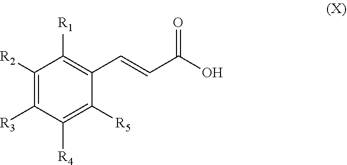

wherein R₁ to R₅ are corresponding to R***, and one among R₁-R₅ is a hydroxyl group and at least one H being in phenolic ortho-position, the rest being H and, optionally an aliphatic alkyl or alkoxy group of C₁-C₆.

Most preferred aliphatic X-hydroxyphenyl acid derivatives may be selected from the group consisting of aliphatic hydroxyphenyl acids (X=1), di-hydroxyphenyl acids (X=2), aliphatic tri-hydroxyphenyl acids (X=3) and aliphatic tetra-hydroxyphenyl acids (X=4), or mixtures thereof, of formula (XI)

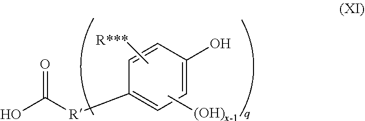

wherein R′ is selected from the group consisting of H, —C-linear or branched C₁-C₆ alkyl or alkoxy group, —C-linear or branched C₂-C₆ alkenyl or alkylenoxy group, —C— substituted or unsubstituted linear or branched C₂-C₆ alkynyl group, and —C-linear or branched C₁-C₆ alkyl or C₂-C₆ alkenyl substituted or unsubstituted phenyl group; and R*** is as defined previously.

The number of R*** in the ring is depending on the number of hydroxyl groups in the ring, and at least one R***, preferably of from 1 to 3, is H towards the phenolic ortho-position, and the integer q is comprised between 1 and 3.

Most preferred diphenolic acid derivatives are of formula (XII)

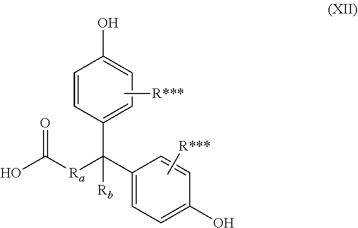

wherein
in said formula, —Rₐ—C—R_{b}— moiety is R′;
on each respective phenolic cycle, at least one R***, preferably of from 1 to 3, is H towards the phenolic ortho-position, and otherwise R*** is as defined previously, and
R_{b} is selected from the group consisting of —(CH₂)ₙ₅CH₃, —(CH₂)ₙ₄-(aliphatic C₁-C₆ aliphatic alkyl or alkoxy substituted or unsubstituted phenyl group), wherein n₅ is an integer from 1 to 12, preferably from 1 to 10, more preferably from 1 to 6, and —(CH₂)ₙ₅(CH(CH₃)₂), and
Rₐ is selected from the group consisting of (CH₂)ₙ₆, wherein n6 is an integer from 1 to 3, —CH(CH₂)ₙ₆(CH₃), —CH(CH(CH₃)₂) and —C(CH₃)₂, —(CH₂)ₙ₆ being the most preferred to lower the steric hindrance.

Most preferred is the 4,4-Bis(4-hydroxyphenyl)valeric acid) (VA or DPA).

The polyfunctional molecule or oligomer compound of formula (III) is of importance for selecting the processing temperature of the benzoxazine polymer.

The compound of formula (III) may advantageously have 1-30, better 1-20, especially 1-10, p values, and may represent more preferably, when Rₚ=H, an ethylene diol with C₁ to C₂₀, preferably C₁ to C₁₂, more preferably C₁ to C₆.

The Bronsted acid type catalyst are those commonly used for a Fischer esterification include para-toluene sulfonic acid (p-TSA), anhydrous chlorhydric acid (HCl), phosphoric acid (H₃PO₄), methanoic acid (CH₃—CO₂H), sulfuric acid, tosylic acid, and Lewis acids such as scandium(III) triflate. The content of catalyst may typically be of from 0.5 wt % to 2 wt %.

The step a) may advantageously be carried out at a temperature in the range of 80° C. to 150° C., most preferably of from 100° C. to 140° C. for the best synthesis yields of higher than 95%, the chosen temperature being dependent on the nature of the reactants, i.e. the melting temperature of said reactant medium.

Advantageously, step a) is performed of from 12 h to 24 h for the highest yield of at least 95%, and the duration is based on the kinetic of the reaction.

The respective stoichiometry of starting reactants on step a), phenolic acid derivative:polyfunctional molecule or oligomer may preferably be 1.0-3.0 eq.:1.0 eq, resulting in an 1.0 eq. of phenol terminated oligomer or molecule.

The second step of the process, step b), corresponds to a Mannich condensation type reaction of the phenol terminated oligomer or molecule of step a) ((IV)) with the amino-alcohol (formula (V)), the primary amine derivative of formula (VI) and the paraformaldehyde (formula (VII)), optionally in presence of a catalyst. Thus, since step b) does not require the use of an external catalyst, step b) is implemented in an easier way.

Advantageously, the amino-alcohol of formula (V) includes R* group, a linear amino-alcohol with a primary amine moiety and an aliphatic hydroxyl moiety for obtaining with the highest yield and the best reaction conditions the oxazine ring.

The amino-alcohol of formula (V) may be more preferably selected from the group consisting of 2-aminoethanol, 2-amino-2-methylpropanol, 5-aminopentan-1-ol, heptaminol and diglycolamine, or mixtures thereof.

The primary amine derivative includes the R** group as defined above.

Primary amine derivatives are the same as R* and may be further selected from the group consisting of allylamine, methylamine, ethylamine, propylamine, butylamine, isopropylamine, hexylamine, cyclohexylamine, 2-aminofluorene, aminophenyl acetylene, propargyl ether aniline, 4-aminobenzonitrile, furfurylamine and aniline, or mixtures thereof.

The temperature range of step b) may preferably be of from 80° C. to 95° C., more allowing to obtain the highest conversion yields of at least 75%.

Advantageously, step b) is performed from 1 h to 8 h, preferably of from 1 h to 5 h, for the highest yield of at least 75%.

One advantage of the invention, is that step b) is performed without any catalyst.

The respective stoichiometry of starting reactants on step b), phenol terminated oligomer or molecule:amino-alcohol:primary amine derivative:paraformaldehyde may preferably be 1.0 eq.:x₁(1.0 eq-18.0 eq):y₁(1.0 eq-18.0 eq):2.0-36.0 eq; or 1.0 eq.:x₂(1.0 eq-18.0 eq):y₂(1.0 eq-18.0 eq):2.0-36.0 eq; or 1.0 eq.:xₚ(1.0 eq-18.0 eq):yₚ(1.0 eq-18.0 eq):2.0-36.0 eq resulting in an 1.0 eq. of the ester-containing benzoxazine monomer, wherein x₁, x₂ and xₚ, independently, =0.1-1, and y₁=1−x₁, y₂=1−x₂ and yₚ=1−xₚ. It is also assumed that the higher are x₁, x₂ and xₚ, independently, the more efficient is the ROP.

The specific range stoichiometry is depending on the respective equivalent proportion of the amino-alcohol and of the primary amine derivative. It should be pointed out that there is a minimal quantity required for the reaction to occur. For instance, the relative molar % of amino-alcohol vs the relative molar % of primary amine derivative is 10 molar % vs 90 molar % respectively. It also means that primary amine may be omitted (0 molar %) and amino-alcohol may only be used instead (100 molar %). Besides, the selected stoichiometry ranges of both amino-alcohol/amine and paraformaldehyde preferably avoids the formation of either reaction linear and/or aliphatic by-products, such as oxazolidine, triaza derivatives, or condensation derivatives.

Preferentially, the whole process is performed with bio-based reactants.

The monoester-benzoxazine synthesis may most preferably be solventless, even though a solvent could be added for the dissolution of starting reactants. The process involves a one-step synthesis, which is one of the advantages of the invention.

Advantageously, the whole synthesis may generally not require any further monomer purification for the invention to be implemented. However, the purification of the monomer, if needed, may be performed by any known technic (vacuum, distillation etc.)

The reaction mixtures of both steps a) and b) are stirred using a classical mechanical stirrer, or any non-limitative means.

The process may be implemented by any known means known to the one skilled in the art, using appropriate vessel either at lab scale or at industrial scale.

The invention also relates to a process for preparing a polybenzoxazine derivative vitrimer comprising the step of polymerization of an ester-containing benzoxazine monomer of the invention (formula (I)) or as obtainable by the above mentioned process at temperatures within the range of from 100° C. to 250° C. for 1 h to 24 h, for obtaining polybenzoxazine derivatives vitrimers.

According to the process for preparing the vitrimers of the invention, the polymerization step, which is a curing step, allows the benzoxazine ring to open and to react on itself to form a 3D network. Once cooled, the shape of the material is kept even after few months, typically 12 months. Once re-heated to at least 100° C. for a few minutes, the ester bonds are exchanging with the aliphatic hydroxyl group allowing the material to be reshaped, recycled, or reprocessed; while keeping structural integrity and number of covalent bound. Considering that Mannich condensation reaction is quantitative, nearly two hydroxyls groups could react with each ester bound through transesterification reaction (even after curing). The vitrimer behaviour strongly depend on the vitrimer glass transition (Tᵥ) also considered as the temperature where the transesterification reaction significantly increased. The vitrimer behaviours were demonstrated through several experiments. After the curing step, by heating the vitrimer above the Tᵥ, an initial shape of the vitrimer can be designed to other original shape. For example, vitrimers may be ground to a powder and can be reshaped or reprocessed at 150° C. in a couple of minutes. However its shape remains stable at room temperature.

The polymerization duration is depending on the curing temperature and/or on the nature of the ester-containing benzoxazine monomer. The polymerization temperature is selected for a given monomer to be higher than the temperature needed to synthesize the monomer. Generally, the higher the polymerization temperature, the shorter the curing duration. For example, when the temperature of the polymerization is 250° C., the curing duration may be of at least 1 h, and for a polymerization temperature of 100° C., the curing duration may be of no more than 24 h. Preferably, the curing temperature may be of from 140° C. to 200° C., more preferably of from 140° C. to 180° C., the latter range providing curing duration of from 1.5 h to 3 h, preferably of from 1.5 h to 2.5 h. The polymerization may be performed by any known heating means, such as laser beam and infrared beam.

The process may also include a post-polymerization step consisting of a heating step which may preferably be carried out at higher temperature than that the polymerization heating step.

The invention is also directed to a polybenzoxazine derivative vitrimer, that may be obtained by the above depicted process, exhibiting at least one of the following characteristics:
(i) Tᵥ values of from −50° C. to 250° C.; preferably of from 130° C. to 220° C., more preferably of from 130° C. to 190° C., and
(ii) Relaxation temperature values, ≥Tᵥ values, of from −50° C. to 300° C., preferably of from 130° C. to 200° C., more preferably of from 130° C. to 180° C.

The vitrimers Tᵥ values are generally dependent from the nature and the content of the catalyst of step b), when present.

The relaxation temperatures typically correspond to the relaxation temperatures of the vitrimers after the appliance of a strain, for example a physical deformation such as a torsion, without the observation of vitrimers degradation.

Advantageously, the vitrimers may also exhibit at least one of the following characteristics selected from the group consisting of:
a relaxation time of from 0.5 s to 2 h, preferably of from 1 s to 1 h, more preferably of from 5 s to 50 min. The relaxation time is conventionally defined as the time for the sample to relax to a value corresponding 1/e (0.37) of its original modulus. Generally, the higher is the temperature, the shorter is the relaxation time. For example, the relaxation time is about 150 min-200 s at temperatures values of 120° C.-170° C., and of s 200, preferably 100 s-20 s, at temperature ranges of 150° C. to 200° C.
In some embodiments, the vitrimer may be deformed between 0.1% to 100% of its initial size;
an activation energy related to relaxation times may be of from 50 kJ/mol to 200 kJ/mol, preferably of from 70 kJ/mol to 170 kJ/mol, more preferably of from 100 kJ/mol to 160 kJ/mol; and
a processing temperature may be of from 100° C. to 250° C., preferably of from 130° C. to 250° C., more preferably of from 150° C. to 200° C., most preferably of from 150° C. to 170° C.

The vitrimers according to the invention may also very preferably exhibit the characteristics of behaving as a thermoset and/or an insolubility in many solvents, without been limited, such as water, CHCl₃, CH₂Cl₂, DMF, THF, aromatic solvents, such as toluene and/or xylene, ketones, alcohols or carboxylic acids. Swelling properties are observed as an extent of from 0 to 500% of the initial weight thereof. Swelling experiments may be carried out in various solvents, for example in acetone, chloroform and water to assess the formation of a cross-linked network. Among them, chloroform is the solvent in which the vitrimer shows the highest swelling ratio of about 100%. In acetone and water, the vitrimers swell of 40%-50% and 20%-30%, respectively.

The vitrimers of the invention present self-healing, reshaping, reprocessability, recycling and reversible adhesive properties.

The vitrimers may constitute an intermediate layer between at least two substrates, such as metal, polymer, glass and ceramic material. The resulting composite material may be prepared by setting at least one ester-containing benzoxazine monomer between the two considered substrates then curing at a temperature providing the vitrimer without altering the integrity of the substrates. Each substrate may be different from the other.

Metallic substrates are not limited, and may be of aluminium, iron, steel and the like.

Polymer substrates may be of polycarbonate, acrylic, polyamide, polyethylene or terephthalate.

Benzoxazine vitrimers may then be advantageously used in non-limited various fields of technologies, such electronics, aerospace, aeronautic, automobile, defense and automotive fields.

The invention also relates to a composition A comprising:
a) an ester-containing benzoxazine derivative of formula (I), and
b) at least one or more additional compounds of organic molecules types containing or not benzoxazine moieties.

Preferably, the organic molecules types may be polymers containing or not benzoxazine moieties.

The additional compound may be used to enhance the properties of either the monomer or the vitrimer (i.e. viscosity, mechanical and thermal properties), or both.

Polymers may be epoxy resins, bismaleimide resins, phenolic resins or benzoxazine resins, polyurethanes, polyamides, polyolefins, polyesters, rubbers. The ester-containing benzoxazine derivative of formula I may be used in a weight ratio from 0.1 to 80% of the final composition.

The compound of formula (I) may be used to provide vitrimer properties to the above mentioned polymers (self-healing, reprocessing, etc.).

The invention also relates to a composition B comprising:
a) an ester-containing benzoxazine monomer of formula (I), and
b) a material selected from the group consisting of fillers, fibers, pigments, dyes, and plasticizer.

The additional compound may be used to enhance the properties of either the monomer or the vitrimer (i.e. viscosity, mechanical and thermal properties), or both.

The additional compound could be carbon fibers, glass fibers, basalt fibers, hemp fibers, and bio-based fibers, clays, carbon black, silica, carbon nanotubes, graphene, any known means for the thermal or the mechanical reinforcement of composites.

The invention also concerns a use of the vitrimer according to the invention as a reversible adhesive, sealant, coating or encapsulating systems for substrates selected from the group consisting of a metal, polymer, glass and ceramic material.

Preferably, the metal and the polymer are as above defined.

The invention also relates to a use of the vitrimer according to the invention in 3D printing processes or in additive manufacturing processes.

Other features and advantages of the present invention will be readily understood from the following detailed description and drawings among them:

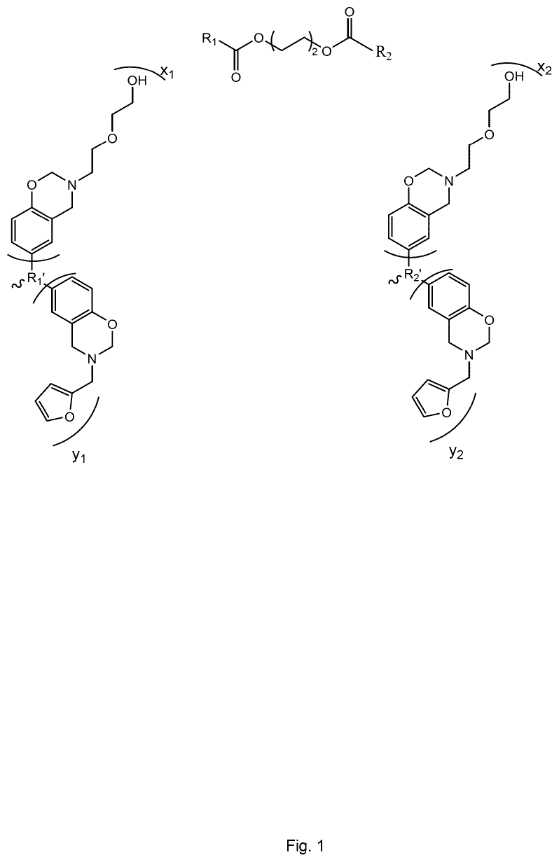

FIG. 1 shows a schematic synthesis reaction for obtaining ester-containing benzoxazine monomer of BUTD-PA-mea/fa type, wherein R₁′ and R₂′ is —CH₂—CH₂— if either x₁ or y₁=0 and if either x₂ or y₂=0, and R₁′ and R₂′ is —CH₂—C(CH₃)— if either x₁ and y₁≠0 and x₂ and y₂≠0, and 0.05≤x₁<1, 0<y₁≤0.95 and 0.05≤x₂<1, 0<y₂≤0.95.

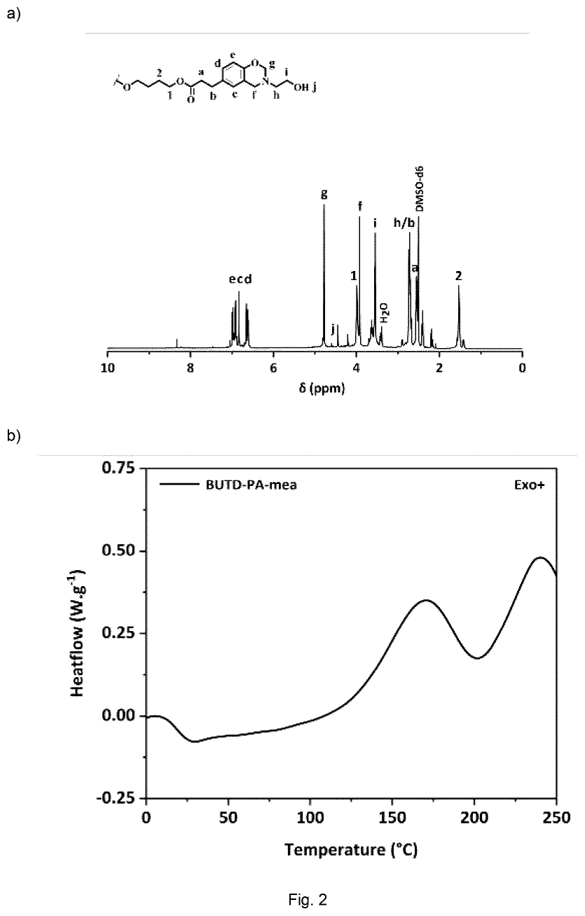

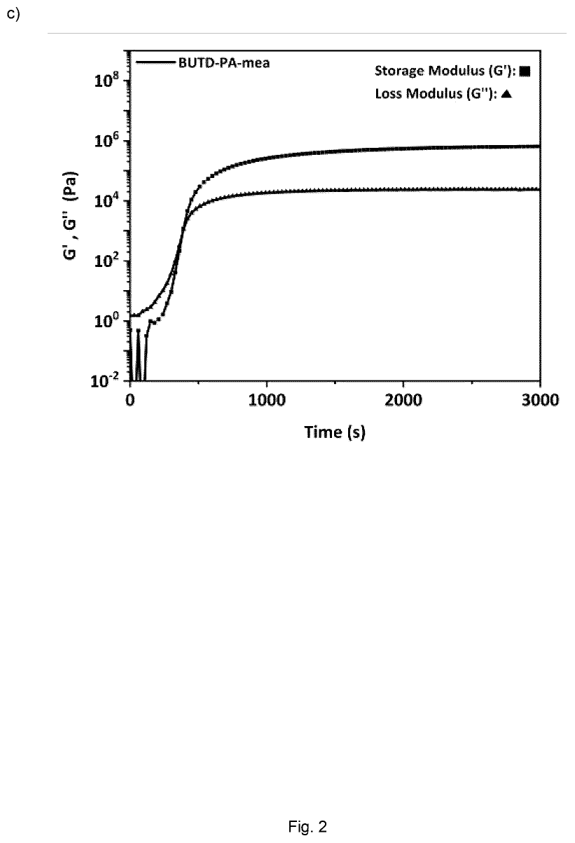

FIG. 2a) displays the NMR spectrum of BUTD-PA-mea ester-containing benzoxazine monomer; FIG. 2b) presents the DSC curve of BUTD-PA-mea; FIG. 2c) presents the isothermal rheology monitoring of BUTD-PA-mea at 140° C.

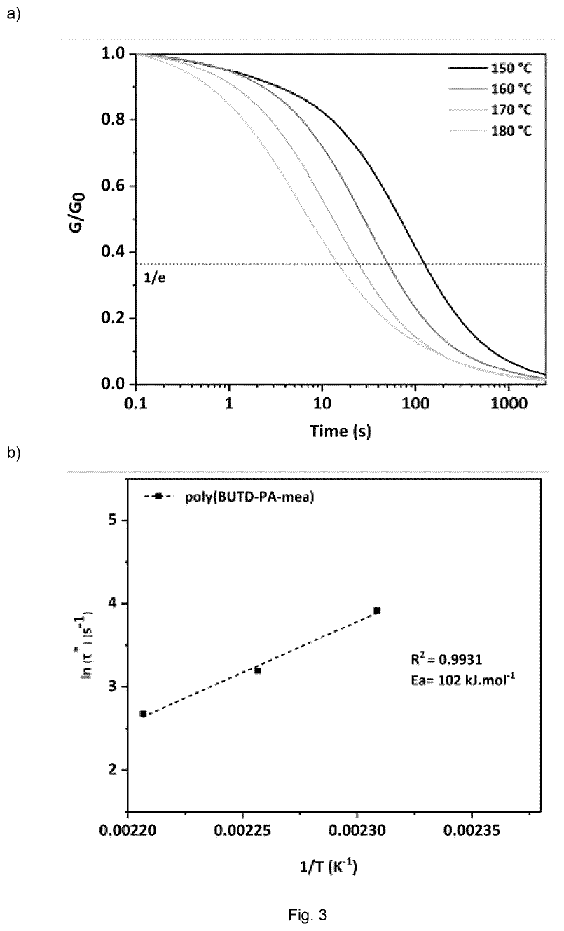

FIG. 3a) shows the Stress relaxation curve of BUTD-PA-mea vitrimer at different temperatures; FIG. 3b) presents the Arrhenius plot obtained from stress relaxation experiment of BUTD-PA-mea vitrimer.

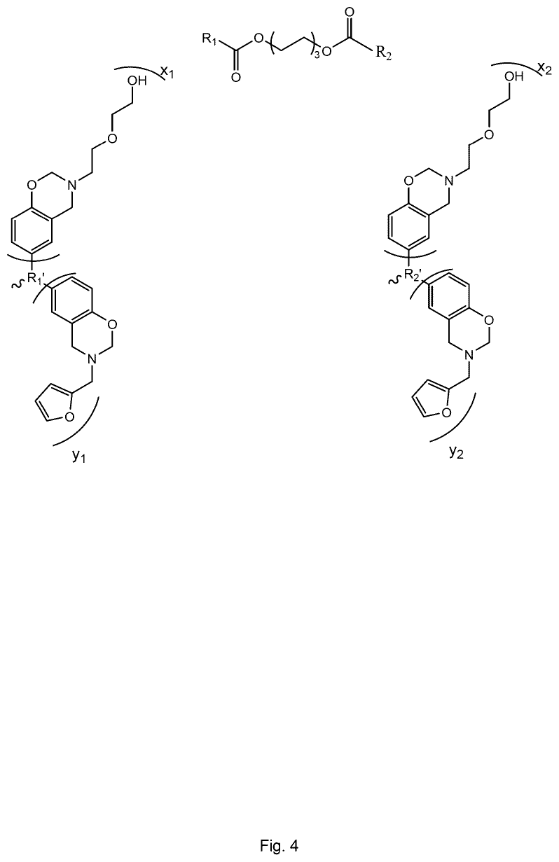

FIG. 4 shows a schematic synthesis reaction for obtaining ester-containing benzoxazine monomer of HEXD-PA-mea/fa type, wherein R₁′ and R₂′ is —CH₂—CH₂— if either x₁ or y₁=0 and if either x₂ or y₂=0, and R₁′ and R₂′ is —CH₂—C(CH₃)— if either x₁ and y₁≠0 and x₂ and y₂≠0, and 0.05≤x₁<1, 0<y₁≤0.95 and 0.05≤x₂<1, 0<y₂≤0.95.

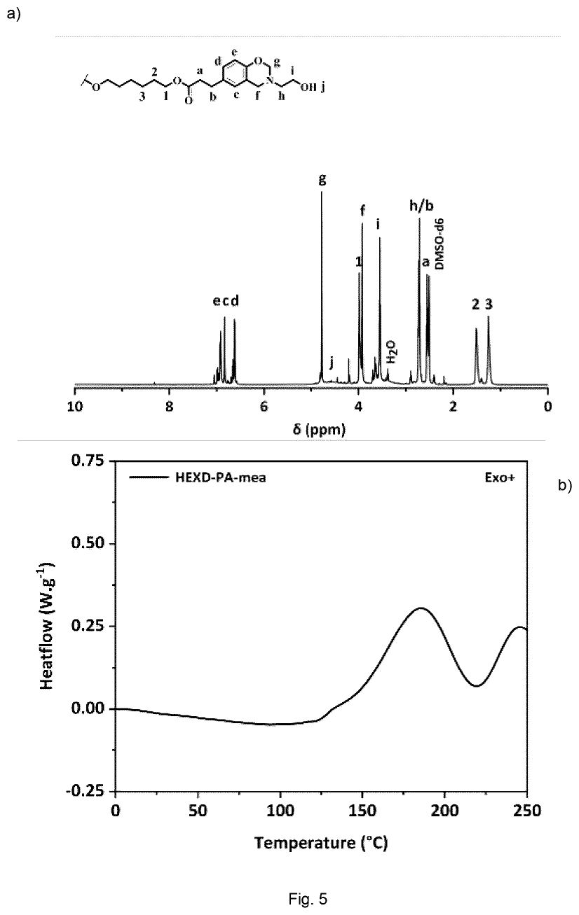

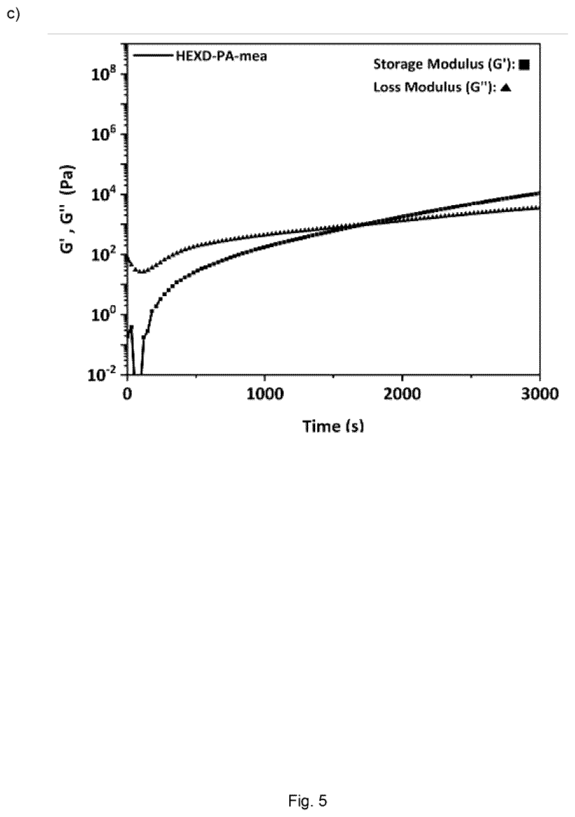

FIG. 5a) displays the NMR spectrum of HEXD-PA-mea ester-containing benzoxazine monomer; FIG. 5b) presents the DSC curve of HEXD-PA-mea; FIG. 5c) presents the isothermal rheology monitoring of HEXD-PA-mea at 140° C.

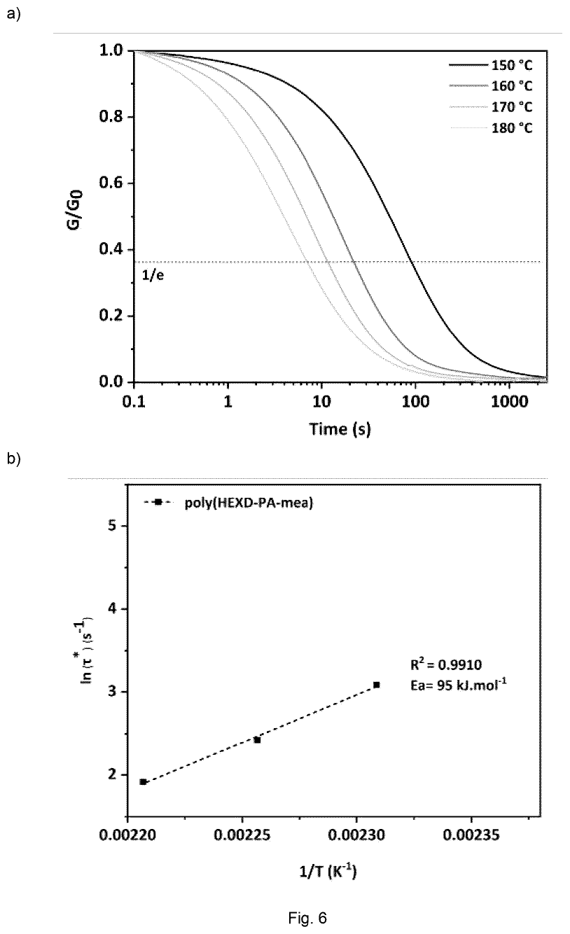

FIG. 6a) shows the Stress relaxation curve of HEXD-PA-mea vitrimer at different temperatures; FIG. 6b) presents the Arrhenius plot obtained from stress relaxation experiment of HEXD-PA-mea vitrimer.

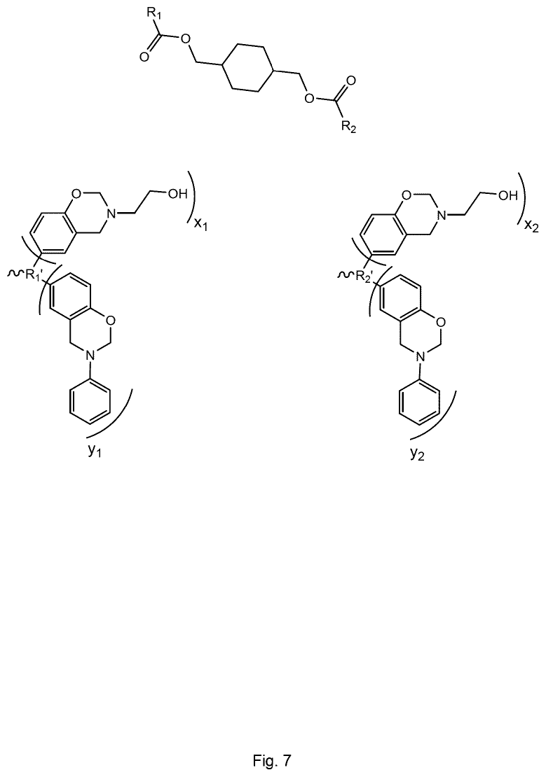

FIG. 7 shows a schematic synthesis reaction for obtaining the ester-containing benzoxazine monomer of CHXD-PA-mea/fa type, wherein R₁′ and R₂′ is —CH₂—CH₂— if either x₁ or y₁=0 and if either x₂ or y₂=0, and R₁′ and R₂′ is —CH₂—C(CH₃)— if either x₁ and y₁≠0 and x₂ and y₂≠0, and 0.05≤x₁<1, 0<y₁≤0.95 and 0.05≤x₂<1, 0<y₂≤0.95.

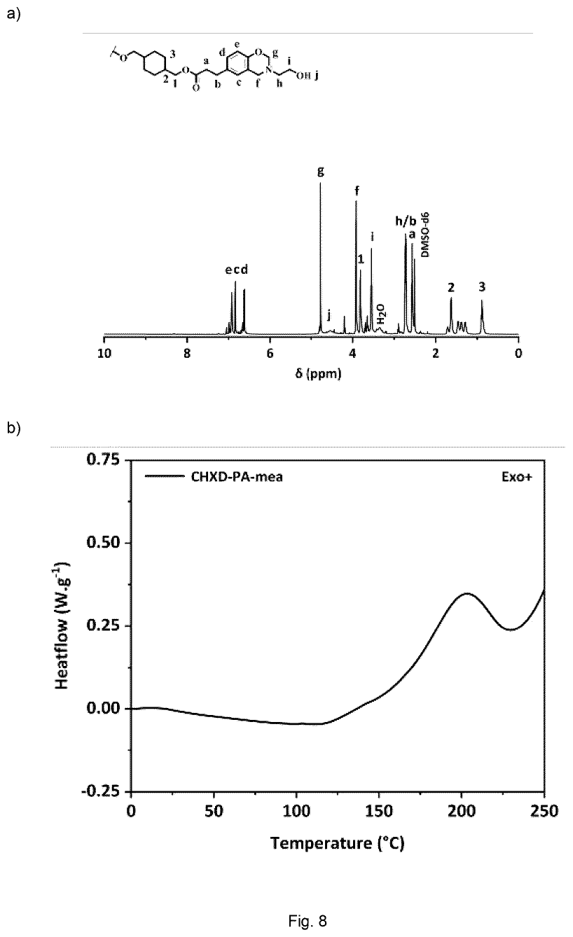

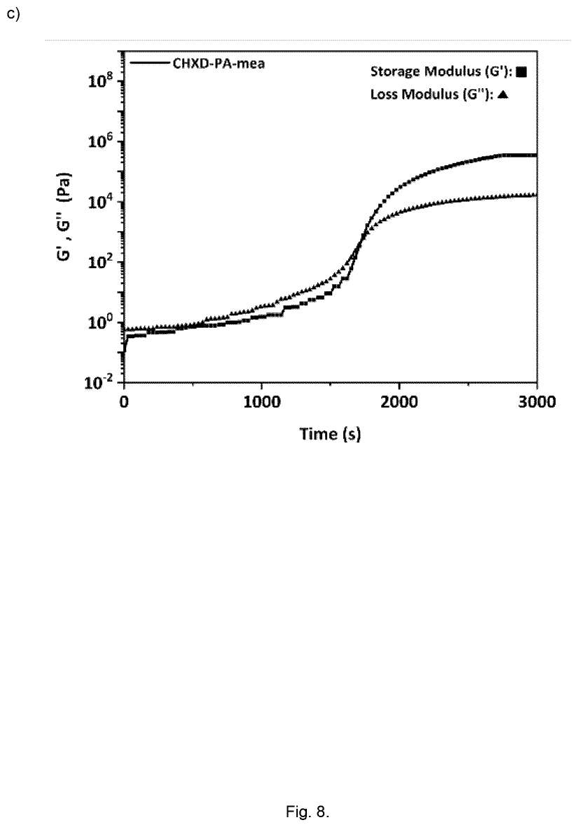

FIG. 8a) displays the NMR spectrum of CHXD-PA-mea ester-containing benzoxazine monomer; FIG. 8b) presents the DSC curve of CHXD-PA-mea; FIG. 8c) presents the isothermal rheology monitoring of CHXD-PA-mea at 140° C.

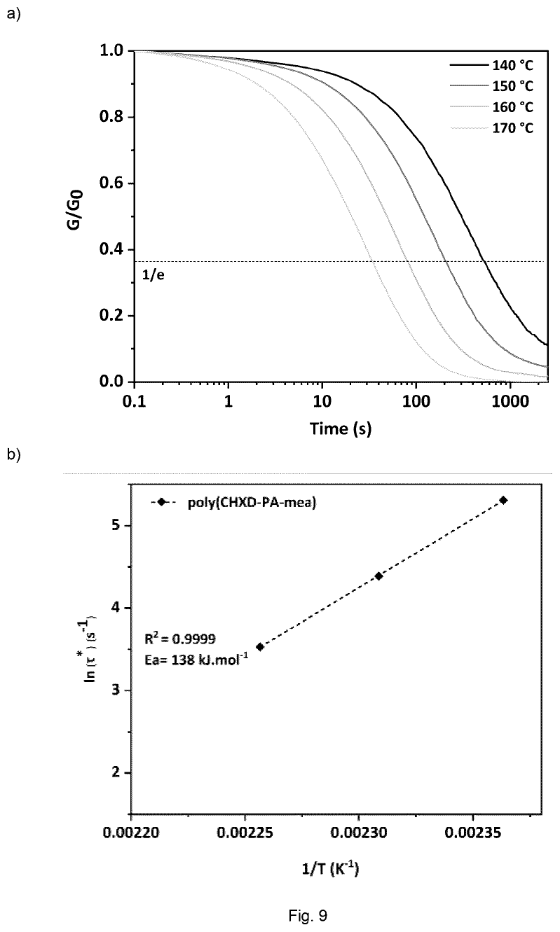

FIG. 9a) shows the Stress relaxation curve of CHXD-PA-mea vitrimer at different temperatures; FIG. 9b) presents the Arrhenius plot obtained from stress relaxation experiment of CHXD-PA-mea vitrimer.

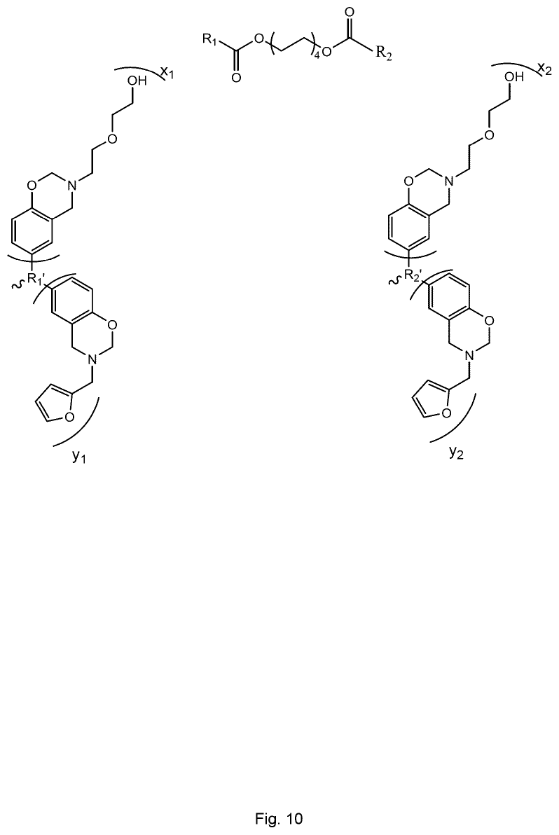

FIG. 10 shows a schematic synthesis reaction for obtaining the ester-containing benzoxazine monomer of OCTD-PA-mea/fa type, wherein R₁′ and R₂′ is —CH₂—CH₂— if either x₁ or y₁=0 and if either x₂ or y₂=0, and R₁′ and R₂′ is —CH₂—C(CH₃)— if either x₁ and y₁≠0 and x₂ and y₂≠0, and 0.05≤x1<1, 0<y₁≤0.95 and 0.05≤x₂<1, 0<y₂≤0.95.

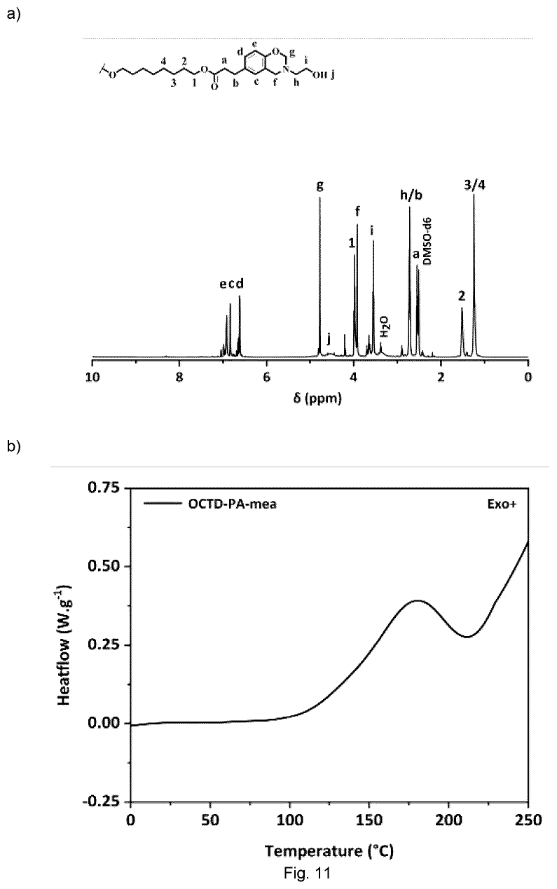

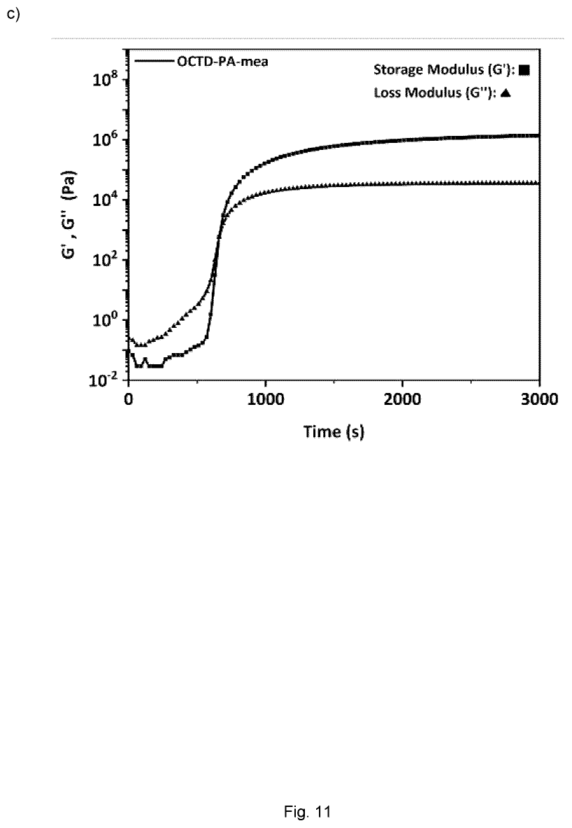

FIG. 11a) displays the NMR spectrum of OCTD-PA-mea ester-containing benzoxazine monomer; FIG. 11b) presents the DSC curve of OCTD-PA-mea; FIG. 11c) presents the isothermal rheology monitoring of OCTD-PA-mea at 140° C.

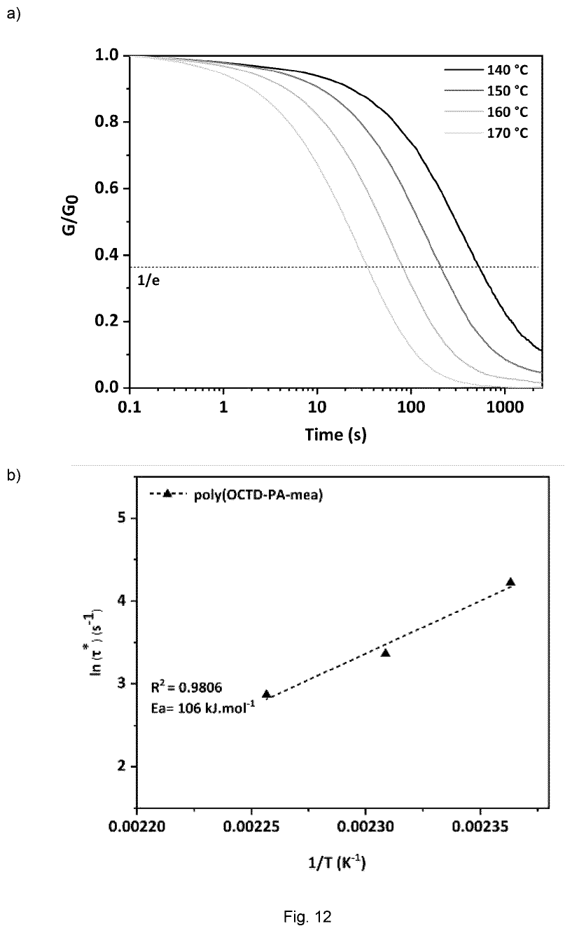

FIG. 12a) shows the stress relaxation curve of OCTD-PA-mea vitrimer at different temperatures; FIG. 12b) presents the Arrhenius plot obtained from stress relaxation experiment of OCTD-PA-mea vitrimer.

All chemicals are commercially available and starting compounds, when applies, used as purchased.

### EXAMPLE 1: SYNTHESIS OF AN ESTER-CONTAINING BENZOXAZINE MONOMER FROM 1,4-BUTANEDIOL (BUTD), 3-(4-HYDROXYPHENYL)PROPANOIC ACID (PA) AS PHENOLIC ACID DERIVATIVES AND ETHANOLAMINE (MEA) AS PRIMARY AMINE WITH ALIPHATIC OH

The first step, step a), corresponds to a Fischer esterification between 1,4-butanediol (M=90.12 g·mol⁻¹, 1 eq, 3 g), 3-(4-Hydroxyphenyl)propanoic acid (PA) (M=166.18 g·mol⁻¹, 2 eq, 11.06 g) in presence of p-toluene sulfonic acid (p-TSA) introduced in catalytic amount (1 wt %). BUTD, PA and p-TSA were reacted together in melt at 110° C. and agitated by mechanical stirring for 24 hours, to provide 3-(4-Hydroxyphenyl)propanoic acid ester terminated 1,4-butanediol (BUTD-PA).

The second step, step b), corresponds to a Mannich condensation between 3-(4-Hydroxyphenyl)-propanoic acid ester terminated BUTD (BUTD-PA) from step a) (M=386.48 g·mol⁻¹, 1 eq, 8 g), ethanolamine (mea) (M=61.08 g·mol⁻¹, 2 eq, 2.53 g) and paraformaldehyde (PFA) (M=30.03 g·mol⁻¹, 4 eq, 2.49 g). All these reactants were reacted together in melt at 85° C. and agitated by mechanical stirring for 8 hours to provide the ester-containing benzoxazine monomer named BUTD-PA-mea (FIG. 1).

The FIG. 2a) displays the NMR spectrum (AVANCE Ill HD Bruker spectrometer) of BUTD-PA-mea ester-containing benzoxazine monomer in DMSO-d₆.

The DSC curve (Netzsch DSC 204 F1 Phoenix apparatus) shows an exothermic peak starting at a temperature of 110° C., with a maximum located at 170° C. (FIG. 2b). This peak corresponds to the ring opening of the benzoxazine rings upon heating. The second peak corresponds to the thermal decomposition of the ester linkage confirmed by TGA experiment.

The approximate gelation (tgel), defined as the crossover point of G′ and G″, was reached quickly after 550 s of isothermal curing at 140° C. (FIG. 2c).

### EXAMPLE 2: VITRIMER SYNTHESIS FROM BUTD-PA-MEA BENZOXAZINE MONOMER

The BUTD-PA-mea benzoxazine monomer was cured 1 h at 150° C., allowing the benzoxazine rings to open and to react on themselves to form a 3D network vitrimer in a disk shape. The vitrimer behaviour of this sample was demonstrated through several rheology experiment.

Viscoelastic properties of BUTD-PA-mea vitrimer were studied by stress relaxation experiments recorded on Anton Paar Physica MCR 302 rheometer in plate-plate mode at 1% shear strain (FIG. 3a). The relaxation time of the polymer was clearly noticeable and was recorded at 2 min at 150° C.

The activation energy (Ea) of transesterification reactions was obtained from the slope of the Arrhenius equation and yielded 102 kJ·mol⁻¹ (FIG. 3b).

### EXAMPLE 3: SYNTHESIS OF AN ESTER-CONTAINING BENZOXAZINE MONOMER FROM 1,6-HEXANEDIOL (HEXD), 3-(4-HYDROXYPHENYL)PROPANOIC ACID (PA) AS PHENOLIC ACID DERIVATIVES AND ETHANOLAMINE (MEA) AS PRIMARY AMINE WITH ALIPHATIC OH

The first step, step a), corresponds to a Fischer esterification between 1,6-hexanediol (M=118.17 g·mol⁻¹, 1 eq, 3 g), 3-(4-Hydroxyphenyl)propanoic acid (PA) (M=166.18 g·mol⁻¹, 2 eq, 8.44 g) in presence of p-toluene sulfonic acid (p-TSA) introduced in catalytic amount (1 wt %). HEXD, PA and p-TSA were reacted together in melt at 110° C. and agitated by mechanical stirring for 24 hours, to provide 3-(4-Hydroxyphenyl)propanoic acid ester terminated 1,6-hexanediol (HEXD-PA).

The second step, step b), corresponds to a Mannich condensation between 3-(4-Hydroxyphenyl)propanoic acid ester terminated HEXD (HEXD-PA) from step a) (M=414.53 g·mol⁻¹, 1 eq, 8 g), ethanolamine (mea) (M=61.08 g·mol⁻¹, 2 eq, 2.36 g) and paraformaldehyde (PFA) (M=30.03 g·mol⁻¹, 4 eq, 2.32 g). All these reactants were reacted together in melt at 85° C. and agitated by mechanical stirring for 8 hours to provide the ester-containing benzoxazine monomer named HEXD-PA-mea (FIG. 4).

The FIG. 8a) displays the NMR spectrum (AVANCE III HD Bruker spectrometer) of HEXD-PA-mea ester-containing benzoxazine monomer in DMSO-d₆.

The DSC curve (Netzsch DSC 204 F1 Phoenix apparatus) shows an exothermic peak starting at a temperature of 120° C., with a maximum located at 185° C. (FIG. 5b). This peak corresponds to the ring opening of the benzoxazine rings upon heating. The second peak corresponds to the thermal decomposition of the ester linkage confirmed by TGA experiment.

The approximate gelation (tgel), defined as the crossover point of G′ and G″, was reached quickly after 1550 s of isothermal curing at 140° C. (FIG. 5c).

### EXAMPLE 4: VITRIMER SYNTHESIS FROM HEXD-PA-MEA BENZOXAZINE MONOMER

The HEXD-PA-mea benzoxazine monomer was cured 1 h at 150° C., allowing the benzoxazine rings to open and to react on themselves to form a 3D network vitrimer in a disk shape. The vitrimer behaviour of this sample was demonstrated through several rheology experiment.

Viscoelastic properties of HEXD-PA-mea vitrimer were studied by stress relaxation experiments recorded on Anton Paar Physica MCR 302 rheometer in plate-plate mode at 1% shear strain (FIG. 6a). The relaxation time of the polymer was clearly noticeable and was recorded at 88 s at 150° C.

The activation energy (Ea) of transesterification reactions was obtained from the slope of the Arrhenius equation and yielded 95 kJ·mol⁻¹ (FIG. 6b).

### EXAMPLE 5: SYNTHESIS OF AN ESTER-CONTAINING BENZOXAZINE MONOMER FROM 1,4-CYCLOHEXANEDIMETHANOL (CHXD), 3-(4-HYDROXYPHENYL)PROPANOIC ACID (PA) AS PHENOLIC ACID DERIVATIVES AND ETHANOLAMINE (MEA) AS PRIMARY AMINE WITH ALIPHATIC OH

The first step, step a), corresponds to a Fischer esterification between 1,4-cyclohexanedimethanol (M=144.21 g·mol-, 1 eq, 3 g), 3-(4-Hydroxyphenyl)-propanoic acid (PA) (M=166.18 g·mol-, 2 eq, 6.91 g) in presence of p-toluene sulfonic acid (p-TSA) introduced in catalytic amount (1 wt %). CHXD, PA and p-TSA were reacted together in melt at 110° C. and agitated by mechanical stirring for 24 hours, to provide 3-(4-Hydroxyphenyl)propanoic acid ester terminated 1,4-cyclohexanedimethanol (CHXD-PA).

The second step, step b), corresponds to a Mannich condensation between 3-(4-Hydroxyphenyl)propanoic acid ester terminated CHXD (HEXD-PA) from step a) (M=440.57 g·mol⁻¹, 1 eq, 8 g), ethanolamine (mea) (M=61.08 g·mol⁻¹, 2 eq, 2.22 g) and paraformaldehyde (PFA) (M=30.03 g·mol⁻¹, 4 eq, 2.18 g). All these reactants were reacted together in melt at 85° C. and agitated by mechanical stirring for 8 hours to provide the ester-containing benzoxazine monomer named CHXD-PA-mea (FIG. 7).

The FIG. 8a) displays the NMR spectrum (AVANCE Ill HD Bruker spectrometer) of CHXD-PA-mea ester-containing benzoxazine monomer in DMSO-d₆.

The DSC curve (Netzsch DSC 204 F1 Phoenix apparatus) shows an exothermic peak starting at a temperature of 120° C., with a maximum located at 185° C. (FIG. 8b). This peak corresponds to the ring opening of the benzoxazine rings upon heating. The second peak corresponds to the thermal decomposition of the ester linkage confirmed by TGA experiment.

The approximate gelation (tgel), defined as the crossover point of G′ and G″, was reached quickly after 1700 s of isothermal curing at 140° C. (FIG. 8c).

### EXAMPLE 6: VITRIMER SYNTHESIS FROM CHXD-PA-MEA BENZOXAZINE MONOMER

The CHXD-PA-mea benzoxazine monomer was cured 1 h at 150° C., allowing the benzoxazine rings to open and to react on themselves to form a 3D network vitrimer in a disk shape. The vitrimer behaviour of this sample was demonstrated through several rheology experiment.

Viscoelastic properties of CHXD-PA-mea vitrimer were studied by stress relaxation experiments recorded on Anton Paar Physica MCR 302 rheometer in plate-plate mode at 1% shear strain (FIG. 9a). The relaxation time of the polymer was clearly noticeable and was recorded at 202 s at 150° C.

The activation energy (Ea) of transesterification reactions was obtained from the slope of the Arrhenius equation and yielded 139 kJ·mol⁻¹ (FIG. 9b).

### EXAMPLE 7: SYNTHESIS OF AN ESTER-CONTAINING BENZOXAZINE MONOMER FROM 1,8-OCTANEDIOL (OCTD), 3-(4-HYDROXYPHENYL)PROPANOIC ACID (PA) AS PHENOLIC ACID DERIVATIVES AND ETHANOLAMINE (MEA) AS PRIMARY AMINE WITH ALIPHATIC OH

The first step, step a), corresponds to a Fischer esterification between 1,8-octanediol (M=146.22 g·mol⁻¹, 1 eq, 3 g), 3-(4-Hydroxyphenyl)propanoic acid (PA) (M=166.18 g·mol⁻¹, 2 eq, 6.91 g) in presence of p-toluene sulfonic acid (p-TSA) introduced in catalytic amount (1 wt %). OCTD, PA and p-TSA were reacted together in melt at 110° C. and agitated by mechanical stirring for 24 hours, to provide 3-(4-Hydroxyphenyl)propanoic acid ester terminated 1,8-octanediol (OCTD-PA).

The second step, step b), corresponds to a Mannich condensation between 3-(4-Hydroxyphenyl)propanoic acid ester terminated OCTD (OCTD-PA) from step a) (M=442.59 g·mol⁻¹, 1 eq, 8 g), ethanolamine (mea) (M=61.08 g·mol⁻¹, 2 eq, 2.21 g) and paraformaldehyde (PFA) (M=30.03 g·mol⁻¹, 4 eq, 2.17 g). All these reactants were reacted together in melt at 85° C. and agitated by mechanical stirring for 8 hours to provide the ester-containing benzoxazine monomer named OCTD-PA-mea (FIG. 10).

The FIG. 11a) displays the NMR spectrum (AVANCE Ill HD Bruker spectrometer) of OCTD-PA-mea ester-containing benzoxazine monomer in DMSO-d₆.

The DSC curve (Netzsch DSC 204 F1 Phoenix apparatus) shows an exothermic peak starting at a temperature of 100° C., with a maximum located at 180° C. (FIG. 12b). This peak corresponds to the ring opening of the benzoxazine rings upon heating. The second peak corresponds to the thermal decomposition of the ester linkage confirmed by TGA experiment.

The approximate gelation (tgel), defined as the crossover point of G′ and G″, was reached quickly after 640 s of isothermal curing at 140° C. (FIG. 11c).

### EXAMPLE 8: VITRIMER SYNTHESIS FROM OCTD-PA-MEA BENZOXAZINE MONOMER

The OCTD-PA-mea benzoxazine monomer was cured 1 h at 150° C., allowing the benzoxazine rings to open and to react on themselves to form a 3D network vitrimer in a disk shape. The vitrimer behaviour of this sample was demonstrated through several rheology experiment.

Viscoelastic properties of OCTD-PA-mea vitrimer were studied by stress relaxation experiments recorded on Anton Paar Physica MCR 302 rheometer in plate-plate mode at 1% shear strain (FIG. 12a). The relaxation time of the polymer was clearly noticeable and was recorded at 68 s at 150° C.

The activation energy (Ea) of transesterification reactions was obtained from the slope of the Arrhenius equation and yielded 106 kJ·mol⁻¹ (FIG. 12b).

1.-14. (canceled)

15. An ester containing benzoxazine monomer of formula (I) 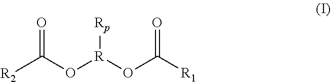
wherein R is selected from the group consisting of a linear or branched C₁-C₂₀ alkyl group, optionally including an heteroatom, a cyclo(C₃-C₆ alkyl) group, a heterocyclo(C₃-C₆ alkyl) group, wherein the heteroatom is selected from the group consisting of from N, S, Si and O, a linear or branched C₂-C₁₂ alkenyl group, a substituted or unsubstituted linear or branched C₂-C₁₂ alkynyl group, a linear or branched C₁-C₆ alkyl or C₂-C₆ alkenyl substituted or unsubstituted phenyl group and a —(CH₂)ₙ₃-phenyl group, wherein n3 is an integer from 1 to 10, a siloxane group;
wherein R₁ is 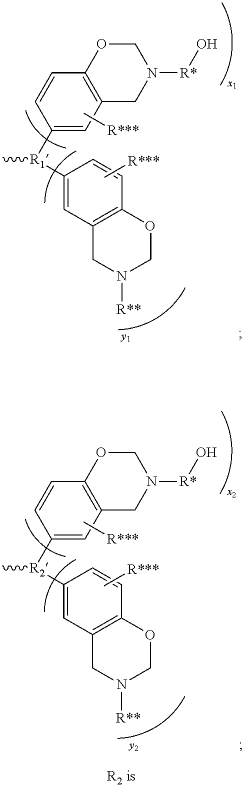
wherein Rₚ is selected from the group consisting of H, a linear or branched C₁-C₂₀ alkyl or alkoxy group, a linear or branched C₂-C₁₂ alkenyl or alkylenoxy group, a substituted or unsubstituted linear or branched C₂-C₁₂ alkynyl group, a linear or branched C₁-C₆ alkyl or C₂-C₆ alkenyl substituted or unsubstituted phenyl group and 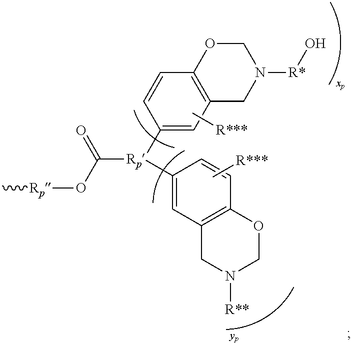
wherein:
R₁ and R₂ of formula (I) are different;
x₁, x₂ and xₚ, independently, are in the range of from 0 to 1; y₁=1−x₁; y₂=1−x₂ yₚ=1−xₚ, with the proviso that x₁, x₂ and xₚ are not together 0; p is 1-100;
wherein R₁′, R₂′, and Rₚ′, independently, are selected from the group consisting of a C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
wherein Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
wherein R* is selected from the group consisting of a linear or branched C₁-C₆ alkyl or alkoxy group, a cyclo(C₃-C₆ alkyl) group, a heterocyclo(C₃-C₆ alkyl) group, wherein the hetero atom is selected form the group consisting of N, Si, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group and a —(CH₂)ₙ₃-phenyl group, —(CH₂)ₙ₃—O—(CH₂)ₙ₄, wherein n3 and n4, independently, are an integer from 1 to 10;
wherein R** is the same as R* and further includes a member selected from a O—, N—, Si— or S—(CH₂)ₙ₃—CH—(CH₃)₂ group, a O—, N—, Si— or S—(CH₂)ₙ₃—(CHZ)ₙ₄—(CH₃)₂ group, a O—, N—, Si— or S—(CH₂)ₙ₃—(CHZ)ₙ₄—(CH₂)ₙ₃—CH₃ group, a O—, N—, Si— or S—(CHZ)ₙ₄—(CH₂)ₙ₃—CH₃ group, a O—, N—, Si— or S—(CHZ)ₙ₄—[(CH₂)ₙ₃—CH₃]₂ group and O-substituted or unsubstituted C₂-C₆ linear or branched alkynyl group, —(CH₂)ₙ₃—C≡N, a polycyclic aromatic or heteroaromatic hydrocarbon, such as naphthalene, anthracene, fluorene, phenanthrene, optionally substituted by a linear or branched C₁-C₆ alkyl or alkoxy group, cyclo(C₃-C₆ alkyl), heterocyclo(C₃-C₆ alkyl), wherein the hetero atom is selected from the group consisting of N, S, Si, and O, linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C, and
wherein R*** is selected from the group consisting of H, OH and O-linear or branched C₁-C₆ alkyl group, and further includes a linear or branched C₁-C₁₅ alkyl group or a C₂-C₁₅ alkenyl group or 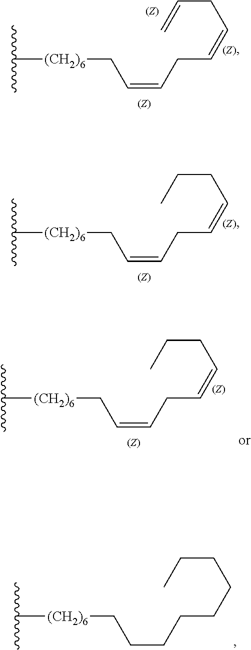
wherein:
$${x}_{1} = \frac{{n}_{aminoalcohol ( R 1 )}}{{n}_{amines ( R 1 )}^{total}}$$
$${x}_{2} = \frac{{n}_{aminoalcohol ( R 2 )}}{{n}_{amines ( R 2 )}^{total}}$$
$${x}_{p} = \frac{{n}_{aminoalcohol ( Rp )}}{{n}_{amines ( Rp )}^{total}}$$
$${y}_{1} = \frac{{n}_{aminoalcohol ( R 1 )}}{{n}_{amines ( R 1 )}^{total}}$$
$${y}_{2} = \frac{{n}_{aminoalcohol ( R 2 )}}{{n}_{amines ( R 2 )}^{total}}$$
$${y}_{p} = \frac{{n}_{aminoalcohol ( Rp )}}{{n}_{amines ( Rp )}^{total}}$$
wherein n_{amine(R1)}^{total}=n_{amines(R1)}+n_{aminoalcohol(R1)}, and n_{aminoalochol(R1)} is the number of aminoalcohol per R₁ group, n_{amines(R1)} represents the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R2)} is the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} is the number of aminoalcohol per Rₚ group, n_{amines(Rp)} represents the number of amines (excepting the number of aminoalcohol) per group Rₚ and n_{amine(Rp)}^{total}+n_{amines(Rp)}+n_{aminoalcohol(Rp)} is the total number of amino groups per group Rₚ.

16. The ester containing benzoxazine monomer according to claim 15,
wherein R* is selected from the group consisting of a linear or branched C₁-C₄ alkyl or alkoxy group, a linear or branched C₂-C₄ alkenyl or alkylenoxy group, an unsubstituted linear or branched C₂-C₄ alkynyl group, an unsubstituted phenyl group and a —(CH₂)ₙ₃-phenyl group, —(CH₂)ₙ₃—O—(CH₂)ₙ₄, wherein n3 and n4, independently, are an integer from 1 to 6;
wherein R** is the same as R* and further includes a member selected from a O—, N—, Si or S—(CH₂)ₙ₃—CH—(CH₃)₂ group, a O—, N—, Si or S—(CH₂)ₙ₃—(CHZ)ₙ₄—(CH₃)₂ group, a O—, N—, Si or S—(CH₂)ₙ₃—(CHZ)ₙ₄—(CH₂)ₙ₃—CH₃ group, a O—, N—, Si or S—(CHZ)ₙ₄—(CH₂)ₙ₃—CH₃ group, a O—, N—, Si or S—(CHZ)ₙ₄—[(CH₂)ₙ₃—CH₃]₂ group and a 0-substituted or unsubstituted C₂-C₄ linear or branched alkynyl group, Z being as defined in claim 1, —(CH₂)ₙ₃—C≡N, cyclo(C₃-C₄ alkyl), heterocyclo(C₃-C₄ alkyl), polycyclic aromatic or heteroaromatic hydrocarbon, wherein the heteroatom is selected from the group consisting of N, S, Si and O, optionally substituted by a linear or branched C₁-C₄ alkyl or alkoxy group, a linear or branched C₂-C₄ alkenyl or alkylenoxy group, or by a substituted or unsubstituted linear or branched C₂-C₄ alkynyl group, wherein n3 and n4, independently, are an integer from 1 to 6; and
wherein R*** is selected from the group consisting of H, OH and a O-linear or branched C₁-C₄ alkyl group, and further includes a linear or branched C₁-C₁₀ alkyl group or C₂-C₁₀ alkenyl group or

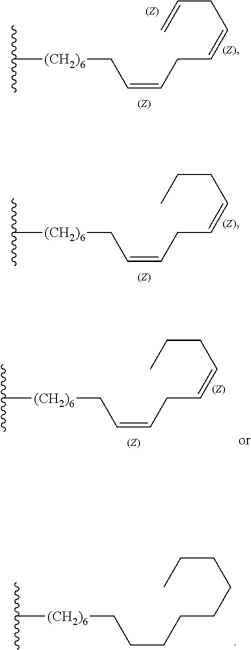

17. The ester containing benzoxazine monomer according to claim 15,
wherein R* is selected from the group consisting of —CH₃, —(CH₂)ₙ₃—CH₃, —(CH₂)ₙ₃—CH—[(CH₂)ₙ₄—CH₃]₂, —C(CH₃)₃, —(CH₂)ₙ₃—(C₆H₅), —(CH₂)ₙ₃—CH═CH₂, —(CH₂)ₙ₃—C≡CH, —(CH₂)ₙ₃—O—(CH₂)ₙ₄ wherein n3 and n4 independently are integer from 1 to 4, phenyl, and —(CH₂)₃-phenyl;
wherein R** is the group R*, or is selected from the group consisting of CH₃, —(CH₂)ₙ₃—CH₃, —(CH₂)ₙ₃—CH—[(CH₂)ₙ₄—CH₃]₂, —C(CH₃)₃, —(CH₂)ₙ₃—(C₆H₅), —(CH₂)ₙ₃—CH═CH₂, —(CH₂)ₙ₃—C≡CH, O—(CH₂)ₙ₃—C≡CH, O—(CH₂)ₙ₃—C≡N, —(CH₂)ₙ₃—C≡N, and —(CH₂)ₙ₃-substituted or unsubstituted furan, phenyl, and wherein n3 and n4, independently, are integer from 1 to 4; and
wherein R*** is selected from the group consisting of H, OH and a O-linear or branched C₁-C₃ alkyl group, and further includes a linear or branched C₁-C₆ alkyl group or C₂-C₆ alkenyl group or

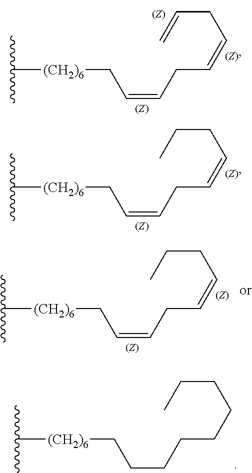

18. A process for synthesizing an ester-containing benzoxazine monomer of formula (I) comprising the following steps consisting of:
a) reacting a phenolic acid derivative of formula (II), comprising at least one R*** group on the phenolic ring: 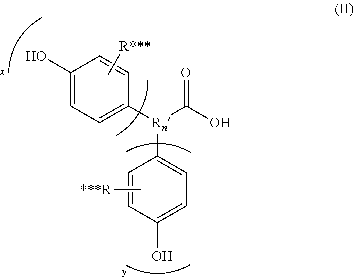
wherein x is of from 0 to 1, and y=1−x,
with a polyfunctional molecule or oligomer of formula (III) 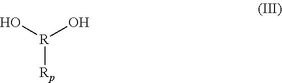
at a temperature of from 25° C. to 200° C., during 1 h-72 h, in the presence of a catalyst of Bronsted acid type, resulting in a phenol terminated oligomer or molecule (compound (IV)), and
b) reacting the compound (IV) with a mixture of:
an amino-alcohol of formula (V): 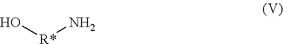
a primary amine derivative of formula (VI),
R**—NH₂ (VI), and
paraformaldehyde of formula (VII) 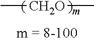
at a temperature range of from 80° C. to 100° C., from 1 h to 10 h, under stirring, for obtaining the compound of formula (I) 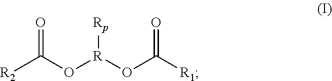
wherein R₁ is 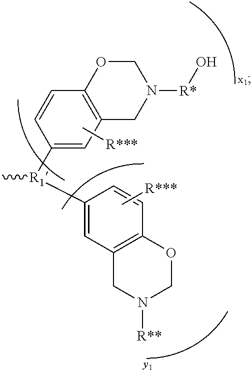
R₂ is 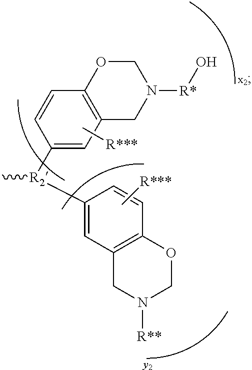
wherein Rₚ is selected from the group consisting of H, a linear or branched C₁-C₂₀ alkyl or alkoxy group, a linear or branched C₂-C₁₂ alkenyl or alkylenoxy group, a substituted or unsubstituted linear or branched C₂-C₁₂ alkynyl group, a linear or branched C₁-C₆ alkyl or C₂-C₆ alkenyl substituted or unsubstituted phenyl group and 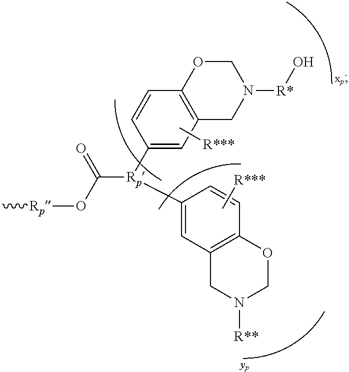
wherein:
R₁ and R₂ of formula (I) are different;
x₁, x₂ and xₚ, independently, are in the range of from 0 to 1; y₁=1−x₁; y₂=1−x₂ yₚ=1−xₚ, with the proviso that x₁, x₂ and xₚ are not together 0; p is 1-100;
wherein R₁′, R₂′, and Rₚ′, independently, are selected from the group consisting of a C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
wherein Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
wherein R* is selected from the group consisting of a linear or branched C₁-C₆ alkyl or alkoxy group, a cyclo(C₃-C₆ alkyl) group, a heterocyclo(C₃-C₆ alkyl) group, wherein the hetero atom is selected form the group consisting of N, Si, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group and a —(CH₂)ₙ₃-phenyl group, —(CH₂)ₙ₃—O—(CH₂)ₙ₄, wherein n3 and n4, independently, are an integer from 1 to 10;
wherein R** is the same as R* and further includes a member selected from a O—, N—, Si— or S—(CH₂)ₙ₃—CH—(CH₃)₂ group, a O—, N—, Si— or S—(CH₂)ₙ₃—(CHZ)ₙ₄—(CH₃)₂ group, a O—, N—, Si— or S—(CH₂)ₙ₃—(CHZ)ₙ₄—(CH₂)ₙ₃—CH₃ group, a O—, N—, Si— or S—(CHZ)ₙ₄—(CH₂)ₙ₃—CH₃ group, a O—, N—, Si— or S—(CHZ)ₙ₄—[(CH₂)ₙ₃—CH₃]₂ group and 0-substituted or unsubstituted C₂-C₆ linear or branched alkynyl group, —(CH₂)ₙ₃—C≡N, a polycyclic aromatic or heteroaromatic hydrocarbon, such as naphthalene, anthracene, fluorene, phenanthrene, optionally substituted by a linear or branched C₁-C₆ alkyl or alkoxy group, cyclo(C₃-C₆ alkyl), heterocyclo(C₃-C₆ alkyl), wherein the hetero atom is selected from the group consisting of N, S, Si, and O, linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C, and
wherein R*** is selected from the group consisting of H, OH and O-linear or branched C₁-C₆ alkyl group, and further includes a linear or branched C₁-C₁₅ alkyl group or a C₂-C₁₅ alkenyl group or 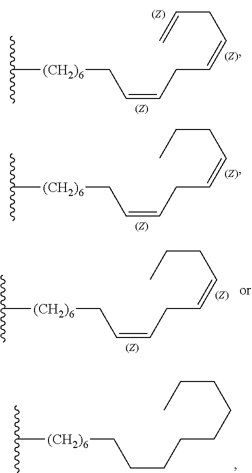
wherein:
$${x}_{1} = \frac{{n}_{aminoalcohol ( R 1 )}}{{n}_{amines ( R 1 )}^{total}}$$
$${x}_{2} = \frac{{n}_{aminoalcohol ( R 2 )}}{{n}_{amines ( R 2 )}^{total}}$$
$${x}_{p} = \frac{{n}_{aminoalcohol ( Rp )}}{{n}_{amines ( Rp )}^{total}}$$
$${y}_{1} = \frac{{n}_{aminoalcohol ( R 1 )}}{{n}_{amines ( R 1 )}^{total}}$$
$${y}_{2} = \frac{{n}_{aminoalcohol ( R 2 )}}{{n}_{amines ( R 2 )}^{total}}$$
$${y}_{p} = \frac{{n}_{aminoalcohol ( Rp )}}{{n}_{amines ( Rp )}^{total}}$$
wherein n_{amine(R1)}^{total}=n_{amines(R1)}+n_{aminoalcohol(R1)}, and n_{aminoalochol(R1)} is the number of aminoalcohol per R₁ group, n_{amines(R1)} represents the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R2)} is the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} is the number of aminoalcohol per Rₚ group, n_{amines(Rp)} represents the number of amines (excepting the number of aminoalcohol) per group Rₚ and n_{amine(Rp)}^{total}+n_{amines(Rp)}+n_{aminoalcohol(Rp)} is the total number of amino groups per group Rₚ,
wherein Rₙ′ is R₁′ or R₂′, R₁′ is different of R₂′, with the proviso that when at least one R*** of the phenolic acid derivative is in ortho position with regard to —OH group, then R*** is H.

19. The process according to claim 18, wherein the phenolic acid derivative (formula (II)) is selected from the group consisting of mono-, di-, tri-hydroxybenzoic acid derivatives, anacardic acid derivatives, hydroxycinnamic acid derivatives, aliphatic X-hydroxyphenyl acid derivatives, wherein X is 2-4, and aliphatic diphenolic acid derivatives, or mixtures thereof.

20. The process according to claim 18, wherein the respective stoichiometry of starting reactants on step a), phenolic acid derivative:polyfunctional molecule or oligomer is 1.0-3.0 eq.:1.0 eq, resulting in an 1.0 eq. of phenol terminated oligomer or molecule.

21. The process according to claim 18, wherein the primary amine derivatives are selected from the group consisting of allylamine, methylamine, ethylamine, propylamine, butylamine, isopropylamine, hexylamine, cyclohexylamine, 2-aminofluorene, aminophenyl acetylene, propargyl ether aniline, 4-aminobenzonitrile, furfurylamine and aniline, or mixtures thereof.

22. The process according to claim 18, wherein the temperature range of step b) is of from 80° C. to 95° C.

23. The process according to claim 18, wherein the step b) is performed from 1 h to 8 h.

24. The process according to claim 18, wherein the respective stoichiometry of starting reactants on step b), phenol terminated oligomer or molecule:amino-alcohol:primary amine derivative:paraformaldehyde is 1.0 eq.:x₁(1.0 eq-18.0 eq):y₁(1.0 eq-18.0 eq):2.0-36.0 eq; or 1.0 eq.:x₂(1.0 eq-18.0 eq):y₂(1.0 eq-18.0 eq):2.0-36.0 eq; or 1.0 eq.:xₚ(1.0 eq-18.0 eq):yₚ(1.0 eq-18.0 eq):2.0-36.0 eq resulting in an 1.0 eq. of the ester-containing benzoxazine monomer, wherein x₁, x₂ and xₚ, independently, =0.1-1, and y₁=1−x₁, y₂=1−x₂ and yₚ=1−xₚ.

25. The process according to claim 18, wherein the relative molar % of amino-alcohol vs the relative molar % of primary amine derivative is 10 molar % vs 90 molar % respectively.

26. A process for preparing a polybenzoxazine derivative vitrimer comprising a step of polymerization of an ester-containing benzoxazine monomer of formula (I) 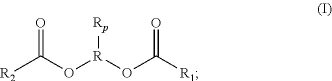
wherein R is selected from the group consisting of a linear or branched C₁-C₂₀ alkyl group, optionally including an heteroatom, a cyclo(C₃-C₆ alkyl) group, a heterocyclo(C₃-C₆ alkyl) group, wherein the heteroatom is selected from the group consisting of from N, S, Si and O, a linear or branched C₂-C₁₂ alkenyl group, a substituted or unsubstituted linear or branched C₂-C₁₂ alkynyl group, a linear or branched C₁-C₆ alkyl or C₂-C₆ alkenyl substituted or unsubstituted phenyl group and a —(CH₂)ₙ₃-phenyl group, wherein n3 is an integer from 1 to 10, a siloxane group;
wherein R₁ is 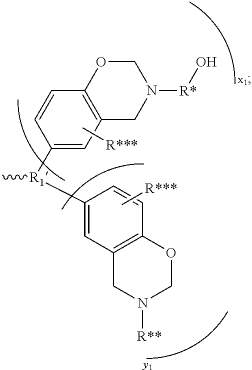
R₂ is 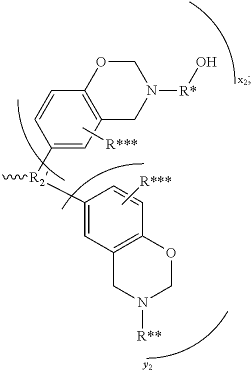
and
wherein Rₚ is selected from the group consisting of H, a linear or branched C₁-C₂₀ alkyl or alkoxy group, a linear or branched C₂-C₁₂ alkenyl or alkylenoxy group, a substituted or unsubstituted linear or branched C₂-C₁₂ alkynyl group, a linear or branched C₁-C₆ alkyl or C₂-C₆ alkenyl substituted or unsubstituted phenyl group and 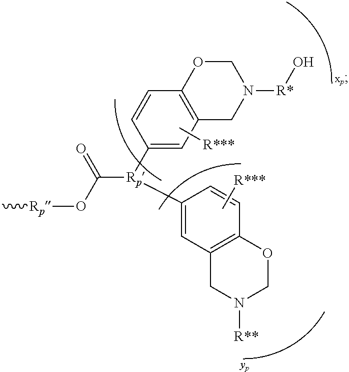
wherein:
R₁ and R₂ of formula (I) are different;
x₁, x₂ and xₚ, independently, are in the range of from 0 to 1; y₁=1−x₁; y₂=1−x₂ yₚ=1−xₚ, with the proviso that x₁, x₂ and xₚ are not together 0; p is 1-100;
wherein R₁′, R₂′, and Rₚ′, independently, are selected from the group consisting of a C-linear or branched C₁-C₆ alkyl or alkoxy group, a —C-linear or branched C₂-C₆ alkenyl or alkylenoxy group, a —C-substituted or unsubstituted linear or branched C₂-C₆ alkynyl group, and a —C-linear or branched C₁-C₆ alkyl or C₂-C₆ alkenyl substituted or unsubstituted phenyl group;
wherein Rₚ″ is selected from the group consisting of a linear or branched C₁-C₆ alkyl or alkoxy group, a linear or branched C₂-C₆ alkenyl or alkylenoxy group, a substituted or unsubstituted linear or branched C₂-C₆ alkynyl group and a linear or branched C₁-C₆ alkyl or C₂-C₆ alkenyl substituted or unsubstituted phenyl group;
wherein R* is selected from the group consisting of a linear or branched C₁-C₆ alkyl or alkoxy group, a cyclo(C₃-C₆ alkyl) group, a heterocyclo(C₃-C₆ alkyl) group, wherein the hetero atom is selected form the group consisting of N, Si, S, and O, a linear or branched C₂-C₆ alkenyl or alkylenoxy group, a substituted or unsubstituted linear or branched C₂-C₆ alkynyl group, a linear or branched C₁-C₆ alkyl or C₂-C₆ alkenyl substituted or unsubstituted phenyl group and a —(CH₂)ₙ₃-phenyl group, —(CH₂)ₙ₃—O—(CH₂)ₙ₄, wherein n3 and n4, independently, are an integer from 1 to 10;
wherein R** is the same as R* and further includes a member selected from a O—, N—, Si— or S—(CH₂)ₙ₃—CH—(CH₃)₂ group, a O—, N—, Si— or S—(CH₂)ₙ₃—(CHZ)ₙ₄—(CH₃)₂ group, a O—, N—, Si— or S—(CH₂)ₙ₃—(CHZ)ₙ₄—(CH₂)ₙ₃—CH₃ group, a O—, N—, Si— or S—(CHZ)ₙ₄—(CH₂)ₙ₃—CH₃ group, a O—, N—, Si— or S—(CHZ)ₙ₄—[(CH₂)ₙ₃—CH₃]₂ group and O-substituted or unsubstituted C₂-C₆ linear or branched alkynyl group, —(CH₂)ₙ₃—C≡N, a polycyclic aromatic or heteroaromatic hydrocarbon, such as naphthalene, anthracene, fluorene, phenanthrene, optionally substituted by a linear or branched C₁-C₆ alkyl or alkoxy group, cyclo(C₃-C₆ alkyl), heterocyclo(C₃-C₆ alkyl), wherein the hetero atom is selected from the group consisting of N, S, Si, and O, linear or branched C₂-C₆ alkenyl or alkylenoxy group, or by a substituted or unsubstituted linear or branched C₂-C₆ alkynyl group, wherein n3 and n4, independently, are an integer from 1 to 10, Z being selected from the group consisting of a linear or branched C₁-C₆ alkyl or alkoxy group, a linear or branched C₂-C₆ alkenyl or alkylenoxy group and a linear or branched C₁-C₆ alkyl or C₂-C₆ alkenyl substituted or unsubstituted phenyl group, and at least one O atom is present or not between two adjacent C,
wherein R*** is selected from the group consisting of H, OH and O-linear or branched C₁-C₆ alkyl group, and further includes a linear or branched C₁-C₁₅ alkyl group or a C₂-C₁₅ alkenyl group or 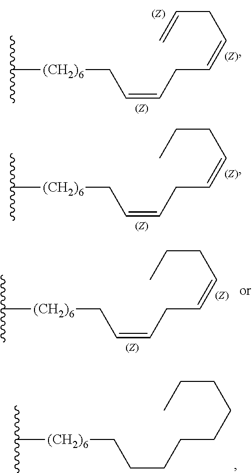
wherein:
$${x}_{1} = \frac{{n}_{aminoalcohol ( R 1 )}}{{n}_{amines ( R 1 )}^{total}}$$
$${x}_{2} = \frac{{n}_{aminoalcohol ( R 2 )}}{{n}_{amines ( R 2 )}^{total}}$$
$${x}_{p} = \frac{{n}_{aminoalcohol ( Rp )}}{{n}_{amines ( Rp )}^{total}}$$
$${y}_{1} = \frac{{n}_{aminoalcohol ( R 1 )}}{{n}_{amines ( R 1 )}^{total}}$$
$${y}_{2} = \frac{{n}_{aminoalcohol ( R 2 )}}{{n}_{amines ( R 2 )}^{total}}$$
$${y}_{p} = \frac{{n}_{aminoalcohol ( Rp )}}{{n}_{amines ( Rp )}^{total}}$$
wherein n_{amine(R1)}^{total}=n_{amines(R1)}+n_{aminoalcohol(R1)}, and n_{aminoalochol(R1)} is the number of aminoalcohol per R₁ group, n_{amines(R1)} represents the number of amines (excepting the number of aminoalcohol) per group R₁ and n_{amine(R1)}^{total}=n_{amines(R1)}+n_{aminoalcohol(R1)} is the total number of amino groups per group R₁;
wherein n_{amine(R2)}^{total}=n_{amines(R2)}+n_{aminoalcohol(R2)}, and n_{aminoalcohol(R2)} is the number of aminoalcohol per R₂ group, n_{amines(R2)} represents the number of amines (excepting the number of aminoalcohol) per group R₂ and n_{amine(R2)}^{total}=n_{amines(R2)}+n_{aminoalcohol(R2)} is the total number of amino groups per group R₂;
wherein n_{amine(Rp)}^{total}=n_{amines(Rp)}+n_{aminoalcohol(Rp)}, and n_{aminoalcohol(Rp)} is the number of aminoalcohol per Rₚ group, n_{amines(Rp)} represents the number of amines (excepting the number of aminoalcohol) per group Rₚ and n_{amine(Rp)}^{total}+n_{amines(Rp)}+n_{aminoalcohol(Rp)} is the total number of amino groups per group Rₚ,
wherein said step of polymerization is at temperatures within the range of from 100° C. to 250° C. for 1 h to 24 h, for obtaining polybenzoxazine derivatives vitrimers.
